(19) United States
(12) Patent Application Publication
(10) Pub. No.: US 2023/0098202 A1
(43) Pub. Date: Mar. 30, 2023

(54) PESTICIDE COMPOSITIONS OF 1-PHENYL-TETRALIN DERIVATIVES

(71) Applicant: METABOLIC INSIGHTS LTD., Ness-Ziona (IL)

(72) Inventors: David PANIK, Rehovot (IL); Ido KORMAN, Sde Nitzan (IL)

(21) Appl. No.: 17/793,998

(22) PCT Filed: Jan. 31, 2021
(86) PCT No.: PCT/IL2021/050110

§ 371 Date: Jul. 20, 2022

Related U.S. Application Data
(60) Provisional application No. 62/969,111, filed on Feb. 2, 2020.

(51) Int. Cl. A01N 31/16; C07C 39/17; C07C 43/23; C07C 211/42; A01N 33/06; A01P 3/00; A01P 1/00

(52) U.S. Cl. CPC A01N 31/16; C07C 39/17; C07C 43/23; C07C 211/42; A01N 33/06; A01P 3/00; A01P 1/00; C07C 2602/10

(57) ABSTRACT

Derivatives of 1-phenyl-tetralin were found to be pesticidally active having high efficiency against several Basidomyceta, Ascomycota and Heterokontophyta fungi as well as protobacteria of the genus Pseudomonas.

### CROSS REFERENCE TO RELATED APPLICATIONS

The present application claims the benefit of priority to U.S. Patent Application No. 62/969,111 filed Feb. 2, 2020, the contents of which are incorporated by reference herein in their entirety.

### FIELD OF THE INVENTION

The present invention relates in general to compounds having fungicidal and bactericidal properties for agricultural uses.

### BACKGROUND OF THE INVENTION

Plant pests and diseases represent major challenges to productivity in modern agriculture. Rusts are a diverse group of plant pathogens with tens of genera and thousands of species. They have huge economic importance and may cause tens of percent's loss in yield in cereals, maize and soybean (Gessese 2019; Groth et al., 1998; Hershman et al., 2011).

Puccinia spp. is an obligatory pathogenic fungus and a major genus in plant rusts belonging to phylogenetic lineage of Basidiomycetes. Puccinia spp. causes a wide range of commercially significant plant diseases in cereals (such as yellow rust in wheat) and maize (common rust)—(Gessese 2019; Groth et al., 1998).

Soil-borne plant pathogens cause crucial damage to agricultural crops. The phytopathogenic fungus Rhizoctonia spp. belongs to phylogenetic lineage of Basidiomycetes. It causes a wide range of commercially significant plant diseases, such as brown patch, damping off in seedlings, root rot and belly rot in vegetable crops and sheath blight in rice. All Rhizoctonia diseases, and subsequent secondary infections in plants are difficult to control (Erlacher et al., 2014).

Pythium spp. is phytopathogenic fungus-like organism which belongs to phylogenetic lineage of eukaryotic microorganisms called Oomycetes which causes the widespread “damping off” disease of tobacco, tomato, mustard, chilies and cress seedlings (Martin & Loper, 2010).

Phytophthora spp. is an obligatory plant fungal like pathogen which belongs to phylogenetic lineage of eukaryotic microorganisms called Oomycetes. Phytophthora infestans is a serious potato disease known as potato blight resulting in foliage blight and rot of tubers. The disease can cause complete loss of a potato harvest (Sedláková et al., 2012). Phytophthora attacks the aerial parts of many plant species and it is the major cause of leaf blight, canker fruit rot diseases in tomato, pumpkins and other crops.

Botrytis spp. is a ubiquitous filamentous fungal pathogen of a wide range of plant species belonging to phylogenetic lineage of Ascomycetes. Botrytis can infect all aerial parts of its host plants to a certain extent. Botrytis causes a disease called grey mold in diverse array of agronomically important crops and commodity plants, such as grapevine, tomato, strawberry, cucumber, bulb flowers, cut flowers and ornamentals (J. A. L. van Kan, 2005).

Fusarium spp. is a large genus of filamentous fungi belonging to phylogenetic lineage of Ascomycetes. Many species of Fusarium are pathogenic to plants and cause serious diseases like wilt or ‘rot’ of economically important plants, mostly vegetables. In addition, Fusarium species infects cereals causing head blight and ear rot in maize and cause to mycotoxins accumulation under certain conditions (J. E. E. Jenkins, Y. S. Clark and A. E. Buckle, 1998).

Alternaria spp. is a ubiquitous fungal genus with numerous species that cause significant damage to agricultural products including cereal grains, fruits and vegetables—apples, potatoes, tomatoes and others (Patriarca, A., & Fernández Pinto, V. 2018).

Pseudomonas spp. is a plant pathogenic bacterial genus which is virulent in the diverse arrays of crop plants and causes to significant leaf and stem lesions. Pseudomonas spp. causes the following diseases in economically significant crops plants and orchards such as: pith necrosis in parsnip and tomato, brown blotch and leaf sheath brown rot in rice, bacterial canker in almonds and olive knot disease in olives (Moore L. W., 1988; Hofte M. and De Vos P., 2006).

A variety of methods have been tested for the management of Pseudomonas spp. in crop plants. They include cultural management, host resistance, biological control with microbial antagonists and chemical control. None of them gives full control.

The number of available active ingredients for crop protection purposes against these diseases is diminishing from year to year due to increasing pest resistance, erratic climatic conditions and mounting regulatory pressure. New active ingredients are urgently needed for development of novel environmentally sustainable crop protection solutions.

### SUMMARY OF INVENTION

In one aspect of the present invention, a method for controlling, preventing, reducing or eradicating instances of plant-pathogen infestation on a plant, plant organ, plant part, or plant propagation material, the method comprises: applying to a plant, plant part, plant organ or plant propagation material, or to soil surrounding said plant, a pesticidally effective amount of at least one compound of formula (I):

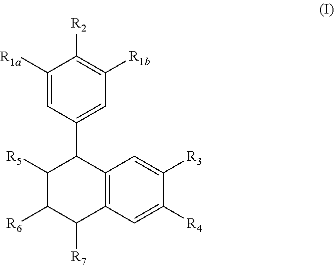

wherein R₁ₐ, R_{1b}, R₂, R₃ and R₄ are independently selected from hydrogen, methyl, hydroxy and methoxy group, and halogen atom (F, Cl, Br, I); R₅ and R₆ are independently selected from hydrogen, methyl and ethyl; and R₇ is selected from hydrogen, methyl, amino, methylamino, dimethylamino, hydroxy and methoxy; or stereoisomers, or agriculturally acceptable salts thereof.

In a specific embodiment, the compounds of formula (I) which are applied in the method of the present invention are:

Compound 3: (1S,4R)-4-(3,4-dichlorophenyl)-N-methyl-1,2,3,4-tetrahydronaphthalen-1-aminium chloride,
Compound 1: (5R,6R,7R)-5-(3,4-dihydroxyphenyl)-6,7-dimethyl-5,6,7,8-tetrahydronaphthalene-2,3-diol, and
Compound 2: 4-((1R,2R,3R)-7-hydroxy-6-methoxy-2,3-dimethyl-1,2,3,4-tetrahydronaphthalen-1-yl)benzene-1,2-diol.

In some embodiments, Compound 3 is applied to a plant-pathogen which is a member selected from: a Basidomycete of the class Pucciniomycetes or the genus Rhizoctonia; an Ascomycota of the class Dothideomycetes or a genus selected from Botrytis and Fusarium; and a Heterokontophyta of the class Oomycota.

In other embodiments, Compound 1 is applied to a plant-pathogen which is a member selected from: a Basidomycete of the class Pucciniomycetes or the genus Rhizoctonia; a Heterokontophyta of the class Oomycota; and a protobacterium of the order Pseudomonadales.

In still other embodiments, Compound 2 is applied to a plant-pathogen which is a member selected from: a Basidomycete of the class Pucciniomycetes or the genus Rhizoctonia; a Heterokontophyta of the family Pythiaceae; and a protobacterium of the order Pseudomonadales.

In another aspect of the present invention, a pesticide composition comprises at least one compound of formula (I),

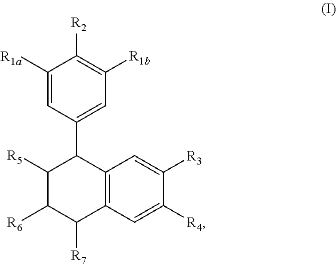

wherein R₁ₐ, R_{1b}, R₂, R₃ and R₄ are independently selected from hydrogen, methyl, hydroxy and methoxy group, and halogen atom (F, Cl, Br, I); R₅ and R₆ are independently selected from hydrogen, methyl and ethyl; and R₇ is selected from hydrogen, methyl, amino, methylamino, dimethylamino, hydroxy and methoxy group; stereoisomers or agriculturally acceptable salts thereof.

In a specific embodiment, the compounds of formula (I) of the composition of the present invention are:

Compound 3: (1S,4R)-4-(3,4-dichlorophenyl)-N-methyl-1,2,3,4-tetrahydronaphthalen-1-aminium chloride,
Compound 1: (5R,6R,7R)-5-(3,4-dihydroxyphenyl)-6,7-dimethyl-5,6,7,8-tetrahydronaphthalene-2,3-diol, and
Compound 2: 4-((1R,2R,3R)-7-hydroxy-6-methoxy-2,3-dimethyl-1,2,3,4-tetrahydronaphthalen-1-yl)benzene-1,2-diol.

### BRIEF DESCRIPTION OF DRAWINGS

FIG. 1 shows the effect of Compound 1 on corn leaf infection by Puccinia sorghi, determined as leaf surface percentage (%) covered by this fungus. ***, p<0.001. ppm, parts per million. (Exp. 343.)

FIGS. 2-3 show the effect of Compound 1 on wheat leaf infection by Puccinia triticina (leaf rust) in two independent experiments, determined as percentage (%) of spore germination (disease severity) 10 days after infection. Signum® (BASF)—a reference fungicide containing 26.7% w/w boscalid and 6.7% w/w pyraclostrobin (the positive control). In Example 9—see composition and preparation of Formulation 1; ***, p<0.001. (Exps. 952 and 973, respectively.)

FIGS. 4-6 show the effect of Compound 3 on corn leaf infection by Puccinia sorghi in three independent experiments, determined as leaf surface percentage (%) covered by the fungus. ***, p<0.001. Formulations 1-5—see Example 10. (Exps. 270, 284 and 294, respectively.)

FIGS. 7-9 show the effect of Compound 3 on Puccinia triticina disease severity of infected wheat plants determined as % of spore germination, using curative approach and spraying application. Formulation 2—see Example 9; *, p<0.05; **, p<0.01; ***, p<0.001; and n.s.—non-significant difference vs. untreated control. (Exps. 135, 153 and 208, respectively.)

FIGS. 10-16 show the effect of Compound 3 on Phytophthora infestans disease severity on tomato plants under greenhouse conditions, determined as % disease severity, using curative approach and application via spraying. Formulation 2—see Example 9; Acrobat® (50% Dimethomorph, BASF); *, p<0.05; **, p<0.01; ***, p<0.001; and n.s.—non-significant difference vs. untreated control. (Exps. 254, 262a, 262b, 275a, 275b, 312a, 312b, respectively.)

FIG. 17 shows the effect of Compound 3 on Alternaria solani disease severity on tomato plants, determined as % disease severity, using preventative approach and application via spraying. Formulation 2—see Example 9; * means that p-value is <0.05; ** means that p-value is <0.01; *** means that p-value is <0.001 and n.s. means non-significant difference vs. untreated control. (Exp. 327).

FIG. 18-19 shows the effect of Compound 3 on Botrytis cinerea disease severity on tomato plants, determined as % disease severity, using preventative approach and application via spraying. Formulations 1 and 2—see Example 9; * means that p-value is <0.05; ** means that p-value is <0.01; *** means that p-value is <0.001 and n.s. means non-significant difference vs. untreated control. (Exps. 314a and 314b, respectively.)

### DETAILED DESCRIPTION OF THE INVENTION

It has been found in accordance with the present invention that 1-phenyl-tetralin derivatives of the following formula (I), stereoisomers or agriculturally acceptable salts thereof are potent pesticides against several Basidomyceta, Ascomycota and Heterokontophyta fungi as well as protobacteria of the genus Pseudomonas:

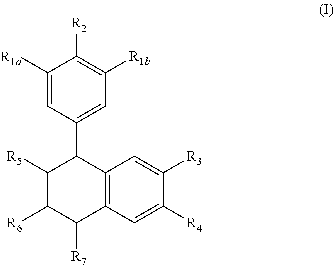

wherein R₁ₐ, R_{1b}, R₂, R₃ and R₄ are independently selected from hydrogen, methyl, hydroxy and methoxy group, and halogen atom (F, Cl, Br, I);

R₅ and R₆ are independently selected from hydrogen, methyl and ethyl; and

R₇ is selected from hydrogen, methyl, amino, methylamino, dimethylamino, hydroxy and methoxy.

In a particular embodiment, the compounds of the present invention are the compounds of formula (I), wherein R₁ₐ, R_{1b}, R₂, R₃ and R₄ are independently selected from hydrogen, methyl, hydroxy and methoxy group, and halogen atom (F, Cl, Br, I); R₅ and R₆ are methyl; and R₇ is selected from hydrogen, methyl, amino, methylamino, dimethylamino, hydroxy and methoxy group.

In a more particular embodiment, the compounds of the present invention are the compounds of formula (I), wherein R₁ₐ, R_{1b}, R₂, R₃ and R₄ are independently selected from hydrogen, hydroxy, and methoxy group; R₅ and R₆ are methyl; and R₇ is selected from hydrogen, methyl, amino, methylamino, dimethylamino, hydroxy and methoxy group.

In a specific embodiment, the compounds of the present invention are the compounds of formula (I), wherein R₁ₐ, R_{1b}, R₂, R₃ and R₄ are independently selected from hydrogen, hydroxy, and methoxy; R₅ and R₆ are methyl; and R₇ is hydrogen.

In the specific embodiment of the present invention, the compounds are:

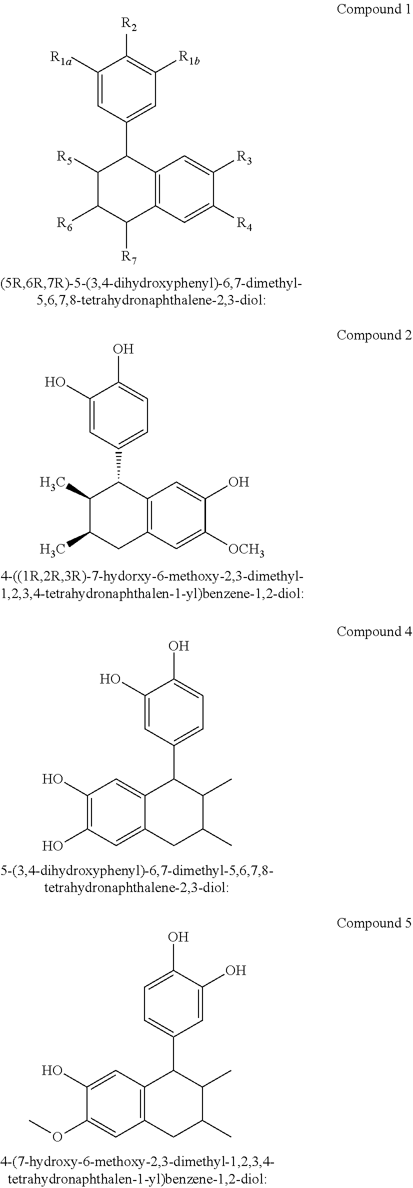

In a further particular embodiment, the compounds of the present invention are the compounds of formula (I), wherein R₁ₐ, R_{1b}, R₂ are independently selected from hydrogen and halogen atom (F, Cl, Br, I); R₃, R₄, R₅ and R₆ are hydrogen; and R₇ is selected from hydrogen, methyl, amino, methylamino, dimethylamino, hydroxy and methoxy group.

In yet further particular embodiment, the compounds of the present invention are the compounds of formula (I), wherein R₁ₐ, R_{1b}, R₂ are independently selected from hydrogen and chlorine atom; R₃, R₄, R₅ and R₆ are hydrogen; and R₇ is methylamino group.

The specific compound of the present invention according to the above embodiment is:

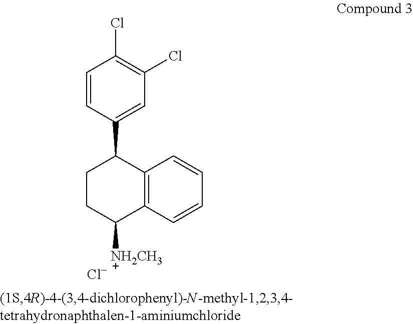

In general, Compound 1 is a 1-phenyl-tetralin derivative, which is a member of the class of 1-aryl tetralin lignans. Compound 2 is another 1-phenyl-tetralin derivative, which is also a member of the class of 1-aryl tetralin lignans. Compound 3 is known as sertraline hydrochloride and it is a selective serotonin reuptake inhibitor (SSRI) anti-depressant drug. These three specific compounds are stereoisomeric 1-phenyl-tetralin derivatives of formula (I).

The present invention provides in one aspect a method for controlling, preventing, reducing or eradicating plant-pathogen infestation or instances thereof, on a plant, plant organ, plant part, or plant propagation material, the method comprising: applying to a plant, plant organ or plant propagation material, or to soil surrounding said plant, a pesticidally effective amount of at least one compound of Compound 3: (1S,4R)-4-(3,4-dichlorophenyl)-N-methyl-1,2,3,4-tetrahydronaphthalen-1-aminium chloride or stereoisomers or agriculturally acceptable salts thereof as an active pesticidal ingredient, or a pesticide composition of compound 3, wherein said plant-pathogen is a member selected from: a Basidomycete of the class Pucciniomycetes or the genus Rhizoctonia; an Ascomycota of the class Dothideomycetes or a genus selected from Botrytis and Fusarium; and; a Heterokontophyta of the class Oomycota.

In another aspect, the present invention provides a method for controlling, preventing, reducing or eradicating instances of plant-pathogen infestation on a plant, plant organ, plant part, or plant propagation material, the method comprising: applying to a plant, plant part, plant organ or plant propagation material, or to soil surrounding said plant, a pesticidally effective amount of Compound 1: (5R,6R,7R)-5-(3,4-dihydroxyphenyl)-6,7-dimethyl-5,6,7,8-tetrahydronaphthalene-2,3-diol, or stereoisomers, or agriculturally acceptable salts thereof, wherein said plant-pathogen is a member selected from: a Basidomycete of the class Pucciniomycetes or the genus Rhizoctonia; a Heterokontophyta of the class Oomycota; and a protobacterium of the order Pseudomonadales.

In additional aspect, the present invention provided a method for controlling, preventing, reducing or eradicating instances of plant-pathogen infestation on a plant, plant organ, plant part, or plant propagation material, the method comprising: applying to a plant, plant part, plant organ or plant propagation material, or to soil surrounding said plant, a pesticidally effective amount of Compound 2: 4-((1R,2R,3R)-7-hydroxy-6-methoxy-2,3-dimethyl-1,2,3,4-tetrahydronaphthalen-1-yl)benzene-1,2-diol, or stereoisomers, or agriculturally acceptable salts thereof, wherein said plant-pathogen is a member selected from: a Basidomycete of the class Pucciniomycetes or the genus Rhizoctonia; a Heterokontophyta of the family Pythiaceae; and a protobacterium of the order Pseudomonadales.

The method of treatment of the present invention according to anyone of the embodiments disclosed herein is useful for example against the following diseases: common rust in corn; crown rust of oats and ryegrass; stem rust of wheat and Kentucky bluegrass, or black rust of cereals; daylily rust; wheat rust in grains; brown or red rust; ‘yellow rust’ in cereals; ‘brown rust’ or ‘orange rust’ in sugarcane; or coffee rust; leaf and stem rust in barley; potato blight, Phytophthora palmivora in cacao, canker fruit rot diseases in tomato and pumpkins; Phytophthora spp. crown and collar rot in pome and stone fruit; “damping off” disease caused by Pythium spp. in tobacco, tomato, cucumbers, mustard, chilies and cress seedlings; gray mold (Botrytis cinerea) in table and wine grapes, strawberries and vegetable crops; Fusarium spp. causing wilt or ‘rot’ of vegetables, bananas; Fusarium spp. head and ear rot in maize; Fusarium graminearum head blight in small grains; Rhizoctonia spp. causing brown patch, damping off in seedlings, root rot and belly rot in vegetables and sheath blight in rice; Alternaria spp. causing spots, rots and blights on leaves and fruits.

In certain embodiments, the plant-pathogen is a member of the class Pucciniomycetes of an order selected from Helicobasidiales, Pachnocybales, Platygloeales, Pucciniales, and Septobasidiales. In specific embodiments, the plant-pathogen is a member of the order Pucciniales.

In some embodiments, the Pucciniales plant-pathogen is a member of a family selected from Chaconiaceae, Coleosporiaceae, Cronartiaceae, Melampsoraceae, Mikronegeriaceae, Phakopsoraceae, Phragmidiaceae, Pileolariaceae, Pucciniaceae, Pucciniosiraceae, Pucciniastraceae, Raveneliaceae, Sphaerophragmiaceae, Uncolaceae, Uropyxidaceae, mitosporic Pucciniales and Pucciniales incertae sedis. In particular embodiments, the Pucciniales plant-pathogen is a member of the family Pucciniaceae.

In other embodiments, the Pucciniaceae plant-pathogen is a member of the genus Puccinia, such as Puccinia sorghi, Puccinia coronate, Puccinia graminis, Puccinia hemerocallidis, Puccinia hemerocallidis, Puccinia persistens subsp. Triticina, Puccinia striiformis, Puccinia melanocephala, Puccinia kuehnii and Hemileia vastatrix. In specific embodiments, the Puccinia plant-pathogen is selected from Puccinia sorghi and Puccinia triticina.

In further embodiments, the method of the invention is useful for controlling, preventing, reducing or eradicating any one of the Pucciniomycetes plant-pathogens described above, and in particular Puccinia sorghi and Puccinia triticina, by applying as described above any one of compounds 3, 1 or 2, or any combination thereof.

In some embodiments, the plant-pathogen is a member of the genus Rhizoctonia (which is in the Ceratobasidiaceae family of the order Cantharellales), such as Rhizoctonia solani, Rhizoctonia bataticola also known as Macrophomina phaseolina, Rhizoctonia carotae also known as Fibulorhizoctonia carotae, Rhizoctonia cerealis, Rhizoctonia crocorum also known as Thanatophytum crocorum, Rhizoctonia fragariae, Rhizoctonia goodyerae-repentis also known as Ceratobasidium cornigerum, Rhizoctonia oryzae also known as Waitea circinate, and Rhizoctonia ramicola also known as Ceratorhiza ramicola. In particular embodiments, the plant-pathogen is Rhizoctonia solani.

In further embodiments, the method of the invention is useful for controlling, preventing, reducing or eradicating any one of the Rhizoctonia plant-pathogens described above, and in particular Rhizoctonia solani, by applying as described above any one of compounds 3, 1 or 2, or any combination thereof.

In yet further embodiments, the plant-pathogen is a member of the class Dothideomycetes of an order selected from Capnodiales, Dothideales, Myriangiales, Hysteriales, Jahnulales, Mytilinidiales, Pleosporales, Botryosphaeriales, Microthyriales, Patellariales, and Trypetheliales. In specific embodiments, the plant-pathogen is a member of the order Pleosporales.

In other embodiments, the Pleosporales plant-pathogen is a member of a family selected from Aigialaceae, Amniculicolaceae, Cucurbitariaceae, Delitschiaceae, Diademaceae, Didymellaceae, Didymosphaeriaceae, Halojulellaceae, Lentitheciaceae, Leptosphaeriaceae, Lindgomycetaceae, Lop hiostomataceae, Massariaceae, Massarinaceae, Melanommataceae, Montagnulaceae, Phaeosphaeriaceae, Phaeotrichaceae, Pleomassariaceae, Pleosporaceae, Sporormiaceae, Venturiaceae, Teichosporaceae, Tetraplosphaeriaceae, Testudinaceae, Trematosphaeriaceae, and Zopfiaceae. In particular embodiments, the Pleosporales plant-pathogen is a member of the family Pleosporaceae.

In some embodiments, the Pleosporaceae plant-pathogen is a member of a genus selected from Alternaria, Bipolaris, Cochliobolus, Crivellia, Decorospora, Exserohilum, Falciformispora, Kriegeriella, Lewia, Macrospora, Monascostroma, Pithomyces, Platysporoides, Pleospora, Pseudoyuconia, Pyrenophora, Setosphaeria, and Zeuctomorpha. In specific embodiments, the Pleosporaceae plant-pathogen is a member of the genus Alternaria.

In still other embodiments, the Alternaria plant-pathogen is selected from Alternaria alternata, Alternaria alternantherae, Alternaria arborescens, Alternaria arbusti, Alternaria blumeae, Alternaria brassicae, Alternaria brassicicola, Alternaria burnsii, Alternaria carotiincultae, Alternaria carthami, Alternaria celosiae, Alternaria cinerariae, Alternaria citri, Alternaria conjuncta, Alternaria cucumerina—grows on various cucurbits, Alternaria dauci, Alternaria dianthi, Alternaria dianthicola, Alternaria eichhorniae, Alternaria euphorbiicola, Alternaria gaisen, Alternaria helianthin, Alternaria helianthicola, Alternaria hungarica, Alternaria infectoria, Alternaria japonica, Alternaria limicola, Alternaria linicola, Alternaria longipes, Alternaria mali, Alternaria molesta, Alternaria panax, Alternaria perpunctulata, Alternaria petroselini, Alternaria porri, Alternaria quercicola, Alternaria radicina, Alternaria raphanin, Alternaria saponariae, Alternaria selini, Alternaria senecionis, Alternaria solani, Alternaria smyrnii, Alternaria tenuissima, Alternaria triticina, Alternaria ventricosa, and Alternaria zinnia. In further specific embodiments, the Alternaria plant-pathogen is selected from Alternaria alternata and Alternaria solani.

In other embodiments, the method of the invention is useful for controlling, preventing, reducing or eradicating any one of the Dothideomycetes plant-pathogens described above, and in particular Alternaria alternata by applying compound 3 as described above; and Alternaria solani, by applying as described above any one of compounds 3, 1 or 2, or any combination thereof.

In still other embodiments, the plant-pathogen is a member of the of the class Leotiomycetes of an order selected from Cyttariales, Erysiphales, Helotiales, Leotiales, and Rhytismatales, Thelebolales.

In further embodiments, the plant-pathogen is a member of the order Helotiales. In yet further embodiments, the Helotiales plant-pathogen is a member of a family selected from Ascocorticiaceae, Dermateaceae, Helotiaceae, Hemiphacidiaceae, Hyaloscyphaceae, Loramycetaceae, Phacidiaceae, Rutstroemiaceae, Sclerotinaceae, and Vibrisseaceae. In particular embodiments, the Helotiales plant-pathogen is a member of the family Sclerotiniaceae. In other particular embodiments, the Sclerotiniaceae plant-pathogen is a member of the genus Botrytis.

In certain embodiments, the Botrytis plant-pathogen is selected from Botrytis aclada, Botrytis allii, Botrytis allii-fistulosi, Botrytis ampelophila, Botrytis anacardii, Botrytis anthophila, Botrytis argillacea, Botrytis arisaemae, Botrytis artocarpi, Botrytis bifurcata, Botrytis bryi, Botrytis capsularum, Botrytis carnea, Botrytis caroliniana, Botrytis carthami, Botrytis cercosporaecola, Botrytis cercosporicola, Botrytis cinerea, Botrytis citricola, Botrytis citrina, Botrytis convallarias, Botrytis croci, Botrytis cryptomeriae, Botrytis densa, Botrytis diospyri, Botrytis elliptica, Botrytis fabae, Botrytis fabiopsis, Botrytis galanthina, Botrytis gladioli, Botrytis gossypina, Botrytis hormini, Botrytis hyacinthi, Botrytis isabellina, Botrytis latebricola, Botrytis liliorum, Botrytis limacidae, Botrytis luteobrunnea, Botrytis lutescens, Botrytis mali, Botrytis monilioides, Botrytis necans, Botrytis paeoniae, Botrytis peronosporoides, Botrytis pistiae, Botrytis platensis, Botrytis pruinosa, Botrytis pseudocinerea, Botrytis pyramidalis, Botrytis rivoltae, Botrytis rosea, Botrytis rubescens, Botrytis rudiculoides, Botrytis sekimotoi, Botrytis septospora, Botrytis setuligera, Botrytis sinoallii, Botrytis sonchina, Botrytis splendida, Botrytis squamosa, Botrytis taxi, Botrytis terrestris, Botrytis tracheiphila, Botrytis trifolii, Botrytis tulipae, Botrytis viciae-hirsutae, and Botrytis yuae. In some embodiments, the plant-pathogen is Botrytis cinerea.

In other embodiments, the plant-pathogen is a member of the class Sordariomycetes of an order selected from Coronophorales, Glomerellales, Hypocreales, Melanosporales, Microascales, Boliniales, Calosphaeriales, Chaetosphaeriales, Coniochaetales, Diaporthales, Magnaporthales, Ophiostomatales, Sordariales, Xylariales, Koralionastetales, Lulworthiales, Meliolales, Phyllachorales, and Trichosphaeriales.

In still other embodiments, the plant-pathogen is a member of the order Hypocreales. In certain embodiments, the Hypocreales plant-pathogen is a member a family selected from Bionectriaceae, Cordycipitaceae, Clavicipitaceae, Hypocreaceae, Nectriaceae, Niessliaceae, Ophiocordycipitaceae, and Stachybotryaceae. In particular embodiments, the Hypocreales plant-pathogen is a member of the family Nectriaceae.

In further embodiments, the Nectriaceae plant-pathogen is a member of the genus Fusarium. In certain embodiments, the Fusarium plant-pathogen is selected from Fusarium acaciae, Fusarium acaciae-mearnsii, Fusarium acutatum, Fusarium aderholdii, Fusarium acremoniopsis, Fusarium affine, Fusarium arthrosporioides, Fusarium avenaceum, Fusarium bubigeum, Fusarium circinatum, Fusarium crookwellense, Fusarium culmorum, Fusarium graminearum, Fusarium incarnatum, Fusarium langsethiae, Fusarium mangiferae, Fusarium merismoides, Fusarium oxysporum, Fusarium pallidoroseum, Fusarium poae, Fusarium proliferatum, Fusarium pseudograminearum, Fusarium redolens, Fusarium sacchari, Fusarium solani, Fusarium sporotrichioides, Fusarium sterilihyphosum, Fusarium subglutinans, Fusarium sulphureum, Fusarium tricinctum, Fusarium venenatum, Fusarium verticillioides, and Fusarium virguliforme. In some embodiments, the plant-pathogen is the species Fusarium oxysporum.

In yet further embodiments, the plant-pathogen is a member of the class Oomycota of an order selected from Lagenidiales, Leptomitales, Peronosporales, Rhipidiales, and Saprolegniales. In certain embodiments, the plant-pathogen is a member of the class Oomycota of the order Peronosporales.

In some embodiments, the Peronosporales plant-pathogen is a member of a family selected from Lagenidiaceae, Olpidiosidaceae, Sirolpidiaceae, Leptomitaceae, Albuginaceae, Peronosporaceae, Pythiaceae, Rhipidaceae, Ectrogellaceae, Haliphthoraceae, Leptolegniellaceae, and Saprolegniaceae. In particular embodiments, the plant-pathogen is a member of the family Peronosporaceae or Pythiaceae.

In certain embodiments, the Peronosporaceae plant-pathogen is a member of a genus selected from Baobabopsis, Basidiophora, Benua, Bremia, Calycofera, Eraphthora, Graminivora, Hyaloperonospora, Nothophytophthora, Novotelnova, Paraperonospora, Perofascia, Peronosclerospora, Peronospora, Phytophthora, Plasmopara, Plasmoverna, Protobremia, Pseudoperonospora, Sclerophthora, Sclerospora, and Viennotia.

In certain embodiments, the Peronosporaceae plant-pathogen is a member of the genus Phytophthora. In specific embodiments, the Phytophthora plant-pathogen is selected from Phytophthora acerina, Phytophthora agathidicida, Phytophthora alni, Phytophthora x alni, Phytophthora alticola, Phytophthora amaranthi, Phytophthora amnicola, Phytophthora amnicola x moyootj, Phytophthora andina, Phytophthora aquimorbida, Phytophthora arecae, Phytophthora arenaria, Phytophthora cf. arenaria, Phytophthora aff arenaria, Phytophthora asiatica, Phytophthora asparagi, Phytophthora aff asparagi, Phytophthora attenuata, Phytophthora austrocedrae, Phytophthora balyanboodja, Phytophthora batemanensis, Phytophthora bilorbang, Phytophthora bisheria, Phytophthora bishii, Phytophthora boehmeriae, Phytophthora boodjera, Phytophthora borealis, Phytophthora botryosa, Phytophthora cf. botryosa, Phytophthora aff. botryosa, Phytophthora brassicae, Phytophthora cactorum, Phytophthora cactorum var. applanata, Phytophthora cactorum x hedraiandra, Phytophthora cajani, Phytophthora cambivora, Phytophthora capensis, Phytophthora capsici, Phytophthora aff. capsici, Phytophthora captiosa, Phytophthora castaneae, Phytophthora castanetorum, Phytophthora chlamydospora, Phytophthora chrysanthemi, Phytophthora cichorii, Phytophthora aff. cichorii, Phytophthora cinnamomi, Phytophthora cinnamomi var. cinnamomi, Phytophthora cinnamomi var. parvispora, Phytophthora cinnamomi var. robiniae, Phytophthora citricola, Phytophthora aff citricola, Phytophthora citrophthora, Phytophthora citrophthora var. clementina, Phytophthora aff citrophthora, Phytophthora clandestina, Phytophthora cocois, Phytophthora colocasiae, Phytophthora condilina, Phytophthora constricta, Phytophthora cooljarloo, Phytophthora crassamura, Phytophthora cryptogea, Phytophthora aff cryptogea, Phytophthora cuyabensis, Phytophthora cyperi, Phytophthora dauci, Phytophthora aff dauci, Phytophthora drechsleri, Phytophthora drechsleri var. cajani, Phytophthora elongata, Phytophthora cf. elongata, Phytophthora erythroseptica, Phytophthora erythroseptica var. pisi, Phytophthora aff erythroseptica, Phytophthora estuarina, Phytophthora europaea, Phytophthora fallax, Phytophthora flexuosa, Phytophthora fluvialis, Phytophthora fluvialis x moyootj, Phytophthora foliorum, Phytophthora formosa, Phytophthora formosana, Phytophthora fragariae, Phytophthora fragariaefolia, Phytophthora frigida, Phytophthora gallica, Phytophthora gemini, Phytophthora gibbosa, Phytophthora glovera, Phytophthora gonapodyides, Phytophthora gondwanensis, Phytophthora gregata, Phytophthora cf. gregata, Phytophthora hedraiandra, Phytophthora aff hedraiandra, Phytophthora x heterohybrida, Phytophthora heveae, Phytophthora hibernalis, Phytophthora himalayensis, Phytophthora himalsilva, Phytophthora aff himalsilva, Phytophthora humicola, Phytophthora aff humicola, Phytophthora hydrogena, Phytophthora hydropathica, Phytophthora idaei, Phytophthora ilicis, Phytophthora x incrassata, Phytophthora infestans, Phytophthora aff infestans, Phytophthora inflata, Phytophthora insolita, Phytophthora cf. insolita, Phytophthora intercalaris, Phytophthora intricata, Phytophthora inundata, Phytophthora ipomoeae, Phytophthora iranica, Phytophthora irrigata, Phytophthora katsurae, Phytophthora kelmania, Phytophthora kernoviae, Phytophthora kwongonina, Phytophthora lactucae, Phytophthora lacustris, Phytophthora lacustris x riparia, Phytophthora lateralis, Phytophthora lilii, Phytophthora litchii, Phytophthora litoralis, Phytophthora litoralis x moyootj, Phytophthora macilentosa, Phytophthora macrochlamydospora, Phytophthora meadii, Phytophthora aff meadii, Phytophthora medicaginis, Phytophthora medicaginis x cryptogea, Phytophthora megakarya, Phytophthora megasperma, Phytophthora melonis, Phytophthora mengei, Phytophthora mexicana, Phytophthora cf. mexicana, Phytophthora mirabilis, Phytophthora mississippiae, Phytophthora morindae, Phytophthora moyootj, Phytophthora moyootj x fluvialis, Phytophthora moyootj x litoralis, Phytophthora moyootj x thermophila, Phytophthora x multiformis, Phytophthora multivesiculata, Phytophthora multivora, Phytophthora nagaii, Phytophthora nemorosa [11], Phytophthora nicotianae, Phytophthora nicotianae var. parasitica, Phytophthora nicotianae x cactorum, Phytophthora niederhauserii, Phytophthora cf. niederhauserii, Phytophthora obscura, Phytophthora occultans, Phytophthora oleae, Phytophthora ornamentata, Phytophthora pachypleura, Phytophthora palmivora, Phytophthora palmivora var. palmivora, Phytophthora parasitica, Phytophthora parasitica var. nicotianae, Phytophthora parasitica var. piperina, Phytophthora parsiana, Phytophthora aff. parsiana, Phytophthora parvispora, Phytophthora x pelgrandis, Phytophthora phaseoli, Phytophthora pini, Phytophthora pinifolia, Phytophthora pisi, Phytophthora pistaciae, Phytophthora plurivora, Phytophthora pluvialis, Phytophthora polonica, Phytophthora porri, Phytophthora primulae, Phytophthora aff. primulae, Phytophthora pseudocryptogea, Phytophthora pseudolactucae, Phytophthora pseudorosacearum, Phytophthora pseudosyringae, Phytophthora pseudotsugae, Phytophthora aff. pseudotsugae, Phytophthora psychrophila, Phytophthora quercetorum, Phytophthora quercina, Phytophthora quininea, Phytophthora ramorum, Phytophthora rhizophorae, Phytophthora richardiae, Phytophthora riparia, Phytophthora rosacearum, Phytophthora aff. rosacearum, Phytophthora rubi, Phytophthora sansomea, Phytophthora sansomeana, Phytophthora aff. sansomeana, Phytophthora x serendipita, Phytophthora sinensis, Phytophthora siskyouensis, Phytophthora sojae, Phytophthora stricta, Phytophthora sulawesiensis, Phytophthora syringae, Phytophthora tabaci, Phytophthora tentaculata, Phytophthora terminalis, Phytophthora thermophila, Phytophthora thermophila x amnicola, Phytophthora thermophila x moyootj, Phytophthora trifolii, Phytophthora tropicalis, Phytophthora cf. tropicalis, Phytophthora tubulina, Phytophthora tyrrhenica, Phytophthora uliginosa, Phytophthora undulata, Phytophthora uniformis, Phytophthora vignae, Phytophthora vignae f. sp. adzukicola, Phytophthora virginiana, and Phytophthora vulcanica. In other specific embodiments, the said plant-pathogen is the species Phytophthora infestans.

In still other embodiments, the Peronosporaceae plant-pathogen is a member of the family Pythiaceae. In certain embodiments, the Pythiaceae plant-pathogen is a member of a genus selected from Cystosiphon, Diasporangium, Globisporangium, Lagenidium, Myzocytium, Phytophthora, Pythium, and Trachysphaera.

In further embodiments, the Pythiaceae plant-pathogen is a member of the genus Pythium. In specific embodiments, the Pythium plant-pathogen is a species selected from Pythium aphanidermatum, Pythium acanthicum, Pythium acanthophoron, Pythium acrogynum, Pythium adhaerens, Pythium amasculinum, Pythium anandrum, Pythium angustatum, Pythium apleroticum, Pythium aquatile, Pythium aristosporum, Pythium arrhenomanes, Pythium attrantheridium, Pythium bifurcatum, Pythium boreale, Pythium buismaniae, Pythium butleri, Pythium camurandrum, Pythium campanulatum, Pythium canariense, Pythium capillosum, Pythium carbonicum, Pythium carolinianum, Pythium catenulatum, Pythium chamaehyphon, Pythium chondricola, Pythium citrinum, Pythium coloratura, Pythium conidiophorum, Pythium contiguanum, Pythium cryptoirregulare, Pythium cucurbitacearum, Pythium cylindrosporum, Pythium cystogenes, Pythium debaryanum, Pythium delicense, Pythium destruens, Pythium diclinum, Pythium dimorphum, Pythium dissimile, Pythium dissotocum, Pythium echinulatum, Pythium emineosum, Pythium erinaceum, Pythium flevoense, Pythium folliculosum, Pythium glomeratum, Pythium graminicola, Pythium grandisporangium, Pythium guiyangense, Pythium helicandrum, Pythium helicoides, Pythium heterothallicum, Pythium hydnosporum, Pythium hypogynum, Pythium indigoferae, Pythium inflatum, Pythium insidiosum, Pythium intermedium, Pythium irregulare, Pythium iwayamae, Pythium jasmonium, Pythium kunmingense, Pythium Morale, Pythium longandrum, Pythium longisporangium, Pythium lutarium, Pythium macrosporum, Pythium mamillatum, Pythium marinum, Pythium marsupium, Pythium mastophorum, Pythium megacarpum, Pythium middletonii, Pythium minus, Pythium monospermum, Pythium montanum, Pythium multisporum, Pythium myriotylum, Pythium nagaii, Pythium nodosum, Pythium nunn, Pythium oedochilum, Pythium okanoganense, Pythium oligandrum, Pythium oopapillum, Pythium ornacarpum, Pythium orthogonon, Pythium ostracodes, Pythium pachycaule, Pythium pachycaule, Pythium paddicum, Pythium paroecandrum, Pythium parvum, Pythium pectinolyticum, Pythium periilum, Pythium periplocum, Pythium perniciosum, Pythium perplexum, Pythium phragmitis, Pythium pleroticum, Pythium plurisporium, Pythium polare, Pythium polymastum, Pythium porphyrae, Pythium prolatum, Pythium proliferatum, Pythium pulchrum, Pythium pyrilobum, Pythium quercum, Pythium radiosum, Pythium ramificatum, Pythium regulare, Pythium rhizo-oryzae, Pythium rhizosaccharum, Pythium rostratifingens, Pythium rostratum, Pythium salpingophorum, Pythium scleroteichum, Pythium segnitium, Pythium speculum, Pythium spinosum, Pythium splendens, Pythium sterilum, Pythium stipitatum, Pythium sulcatum, Pythium terrestris, Pythium torulosum, Pythium tracheiphilum, Pythium ultimum, Pythium ultimum var. ultimum, Pythium uncinulatum, Pythium undulatum, Pythium vanterpoolii, Pythium viniferum, Pythium violae, Pythium volutum, Pythium zingiberis, and Pythium zingiberum. In other specific embodiments, the plant-pathogen is the species Pythium aphanidermatum.

In other embodiments of the present invention, the method of the invention is useful for controlling, preventing, reducing or eradicating any one of the Oomycotaplant-pathogens described above, and in particular Phytophthora infestans by applying compound 3 as described above; and Pythium aphanidermatum, by applying as described above compound 3 or 1, or a combination thereof.

In further embodiments, the plant-pathogen is a member of the genus Pseudomonas, such as Pseudomonas aeruginosa and Pseudomonas syringae. In particular embodiments, the plant-pathogen is the species Pseudomonas syringae. In yet further embodiments, the method of the invention is useful for controlling, preventing, reducing or eradicating any one of the Pseudomonas pathogens described above, and in particular Pseudomonas syringae, by applying as described above compound 1 or 2, or a combination thereof.

In another aspect, the present invention provides a pesticide composition comprising a pesticidally effective amount of at least one compound of formula (I),

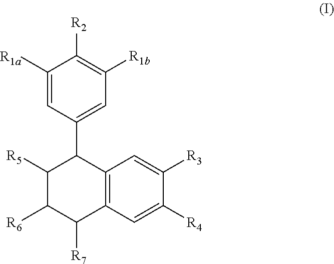

wherein R₁ₐ, R_{1b}, R₂, R₃ and R₄ are independently selected from hydrogen, methyl, hydroxy and methoxy group, and halogen atom (F, Cl, Br, I); R₅ and R₆ are independently selected from hydrogen, methyl and ethyl; and R₇ is selected from hydrogen, methyl, amino, methylamino, dimethylamino, hydroxy and methoxy; stereoisomers or agriculturally acceptable salts thereof.

In a further embodiment, the present invention provides a pesticide composition comprising a compound of formula (I), wherein R₁ₐ, R_{1b}, R₂, R₃ and R₄ are independently selected from hydrogen, methyl, hydroxy and methoxy group, and halogen atom (F, Cl, Br, I); R₅ and R₆ are methyl; and R₇ is selected from hydrogen, methyl, amino, methylamino, dimethylamino, hydroxy and methoxy group.

In yet further embodiment, the present invention provides a pesticide composition comprising a compound of formula (I), wherein R₁ₐ, R_{1b}, R₂, R₃ and R₄ are independently selected from hydrogen, hydroxy, and methoxy group; R₅ and R₆ are methyl; and R₇ is selected from hydrogen, methyl, amino, methylamino, dimethylamino, hydroxy and methoxy group.

In a particular embodiment, the present invention provides a pesticide composition comprising a compound of formula (I), wherein R₁ₐ, R_{1b}, R₂, R₃ and R₄ are independently selected from hydrogen, hydroxy, and methoxy; R₅ and R₆ are methyl; and R₇ is hydrogen.

In a specific embodiment, the pesticide composition of the present invention comprises:

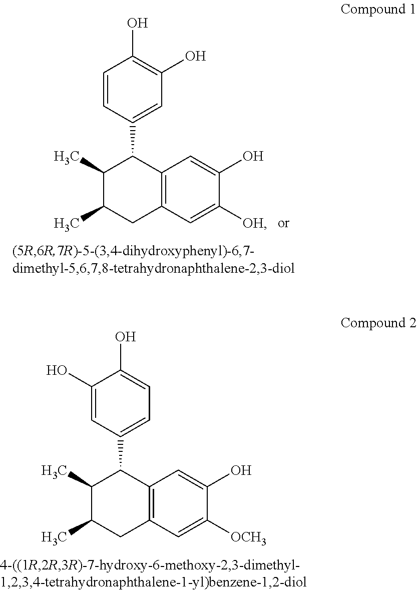 [SMILES: C[C@H]1[C@H](c2ccc(O)c(O)c2)c2cc(O)c(O)cc2C[C@H]1C ; COc1cc2c(cc1O)[C@@H](c1ccc(O)c(O)c1)[C@H](C)[C@H](C)C2]

In another embodiment, the present invention provides a pesticide composition comprising a compound of formula (I), wherein R₁ₐ, R_{1b}, R₂ are independently selected from hydrogen and halogen atom (F, Cl, Br, I); R₃, R₄, R₅ and R₆ are hydrogen; and R₇ is selected from hydrogen, methyl, amino, methylamino, dimethylamino, hydroxy and methoxy group.

In a specific embodiment, the present invention provides a pesticide composition comprising a compound of formula (I), wherein R₁ₐ, R_{1b}, R₂ are independently selected from hydrogen and chlorine atom; R₃, R₄, R₅ and R₆ are hydrogen; and R₇ is methylamino group.

In another specific embodiment, the pesticide composition of the present invention comprises:

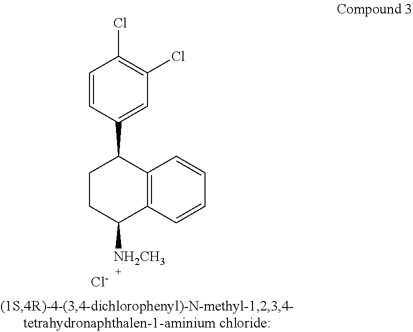

In certain embodiments, the pesticide composition or formulation of any one of the above embodiments further comprises an agriculturally suitable or acceptable solvent or solubilising agent. In other certain embodiments, the agriculturally acceptable solvent or solubilising agent is a water-miscible solvent capable of dissolving or solubilising 1-phenyl-tetralin compounds.

In some embodiments, the water-miscible solvent capable of dissolving or solubilising 1-phenyl-tetralin compounds is a polar solvent, such as an alcohol, a ketone, a lactone, a keto-alcohol, a glycol, a glycoether, an amide, an alkanolamine, a sulfoxide and a pyrrolidone. In particular embodiments, the composition of any one of the above embodiments comprises a solvent selected from dimethyl-sulfoxide or ethanol. In specific embodiments, the composition further comprises a polysorbate-type non-ionic surfactant, such as polysorbate 20.

The pesticide composition of the present invention may be formulated into a formulation to facilitate application of the active pesticidal ingredient. The formulation may be a water-miscible formulation, such as a suspension concentrate (SC), a capsule suspension (CS), water-dispersible granules (WG), an emulsifiable concentrate (EC), a wettable powder (WP), a soluble (liquid) concentrate (SL), or a soluble powder (SP).

The composition or formulation of the present invention may further comprise at least one adjuvant, carrier, diluent, and/or surfactant. Non-limiting examples of adjuvants are activator adjuvants, such as cationic, anionic or non-ionic surfactants, oils and nitrogen-based fertilisers capable of improving activity of the pesticide product. Oils may be crop oils, such as paraffin or naphtha-based petroleum oil, crop oil concentrates based on emulsifiable petroleum-based oil, and vegetable oil concentrates derived from seed oil, usually cotton, linseed, soybean, or sunflower oil, used to control grassy weeds. Nitrogen-based fertilisers may be ammonium sulphate or urea-ammonium nitrate.

A non-limiting example of a polysaccharide adjuvant used also as a thixotropic agent in the compositions of the present embodiments, is Xanthan gum (commercially available under trademark KELZAN® by CP Kelco), which is produced from simple sugars using a fermentation process, and derives its name from the species of bacteria used, Xanthomonas campestris. Oils used as adjuvants may be crop oils, such as paraffin or naphtha-based petroleum oil, crop oil concentrates based on emulsifiable petroleum-based oil, and vegetable oil concentrates derived from seed oil, usually cotton, linseed, soybean, or sunflower oil, used to control grassy weeds. Nitrogen-based fertilisers may be ammonium sulphate or urea-ammonium nitrate.

Non-limiting examples of solubilising agents or solvents are petroleum-based solvents, the aforementioned oils, liquid mixtures of fatty acids, ethanol, glycerol and dimethyl sulfoxide. The agriculturally acceptable solvent or solubilising agent may be a water-miscible solvent capable of dissolving or solubilising 1-phenyl-tetralin compounds, such as a polar solvent, e.g., an alcohol, a ketone, a lactone, a keto-alcohol, a glycol, a glycoether, an amide, an alkanolamine, a sulfoxide and a pyrrolidone. Non-limiting examples of carriers are precipitated silica, colloidal silica, attapulgite, china clay, talc, kaolin and combinations thereof.

The pesticide composition or formulation of the present invention may further comprise a diluent, such as lactose, starch, urea, water soluble inorganic salts and combination thereof. The pesticide composition or formulation may further comprise one or more surfactants, such as polysorbate-type non-ionic surfactant, for example Polysorbate 20 or trisiloxane non-ionic surfactant, styrene acrylic dispersant polymers, acid resin copolymer based dispersing agents, potassium polycarboxylate, sodium alkyl naphthalene sulphonate blend, sodium diisopropyl naphthalene sulphonate, sodium salt of naphthalene sulphonate condensate, lignin sulfonate salts and combinations thereof.

Trisiloxane non-ionic surfactants or polyether dimethyl siloxanes (PEMS), often referred to as super-spreaders or super-wetters, are added to pesticides to enhance their activity and the rain fastness of the active substance by promoting rapid spreading over the hydrophobic surfaces of leaves. Some spreaders of the modified trisiloxane type combine a very low molecular weight trisiloxane with a polyether group and capable of reducing surface tension and rapidly spreading on difficult to wet surfaces.

The active agent, composition, or formulation comprising it, is applied in the method of any one of the above embodiments to the plant or part, organ or plant propagation material thereof by spraying, immersing, dressing, coating, pelleting or soaking.

In certain embodiments, the concentration of the 1-phenyl-tetralin compounds of the present invention, in the composition or formulation comprising it may be in the range of 10-2000, 10-1500, 10-1000, 10-900, 10-800, 10-700, 10-600, 10-500, 10-400, 10-300, 10-200, 10-900, 20-800, 20-700, 20-600, 20-500, 20-400, 20-300, 20-200, 20-100, 20-90, 20-80, 20-70, 20-60, 20-50, 20-40, 20-30, 20-20. 30-2000, 30-1500, 30-1000, 30-900, 30-800, 30-700, 30-600, 30-500, 30-400, 30-300, 30-200, 30-100, 30-0, 30-100, 30-90, 30-80, 30-70, 30-60, 30-50, 30-40, 40-2000, 40-1500, 40-1000, 40-900, 40-800, 40-700, 40-600, 40-500, 40-400, 40-300, 40-200, 40-100, 40-90, 40-80, 40-70, 40-60, 40-50, 50-2000, 50-1500, 50-1000, 50-900, 50-800, 50-700, 50-600, 50-500, 50-400, 50-300, 50-200, 50-100, 50-90, 50-80, 50-70, 50-60, 60-2000, 60-1500, 60-1000, 60-900, 60-800, 60-700, 60-600, 60-500, 60-400, 60-300, 60-200, 60-100, 60-90, 60-80, 60-70, 70-2000, 70-1500, 70-1000, 70-900, 70-800, 70-700, 70-600, 70-500, 70-400, 70-300, 70-200, 70-100, 70-90, 70-80, 80-2000, 80-1500, 80-1000, 80-900, 80-800, 80-700, 80-600, 80-500, 80-400, 80-300, 80-200, 80-100, 80-90, 90-2000, 90-1500, 90-1000, 90-900, 90-800, 90-700, 90-600, 90-500, 90-400, 90-300, 90-200, 90-100, 100-2000, 100-1500, 100-1000, 100-900, 100-800, 100-700, 100-600, 100-500, 100-400, 100-300, 100-200, 200-2000, 200-1500, 200-1000, 200-900, 200-800, 200-700, 200-600, 200-500, 200-400, 200-300, 300-2000, 300-1500, 300-1000, 300-900, 300-800, 300-700, 300-600, 300-500, 300-400, 400-2000, 400-1500, 400-1000, 400-900, 400-800, 400-700, 400-600, 400-500, 500-2000, 500-1500, 500-1000, 500-900, 500-800, 500-700, 500-600, 600-2000, 600-1500, 600-1000, 600-900, 600-800, 600-700, 700-2000, 700-1500, 700-1000, 700-900, 700-800, 800-2000, 800-1500, 800-1000, 800-900, 900-2000, 900-1500, 900-1000, 1000-2000, or 1000-1500 ppm.

In particular, the concentration of 1-phenyl-tetralin compounds in the composition or formulation comprising it may be 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 1000, 1500 or 2000 ppm.

Any one of the above concentration ranges or concentrations can be used in accordance with any one of the above embodiments of the method of the present invention, including against any one of the aforementioned pathogens and by means of any one of the above mentioned means of applying the composition or formulation.

### Definitions

The term “plant organ” as used herein refers to the leaf, stem, root, and reproductive structures. The term “plant part” as used herein refers to a vegetative plant material such as a cutting or a tuber; a leaf, flower, bark or a stem. The term “plant propagation material” as used herein refers to a seed, root, fruit, tuber, bulb, rhizome, or part of a plant. The term “pesticidal effective amount” as used herein refers to an amount of the pesticide that is able to bring about death to at least one pest, or to noticeably reduce pest growth, feeding, or normal physiological development. The terms “class”, “order”, “family”, “genus”, and “species” are used herein according to Art 3.1 of the International Code of Nomenclature for algae, fungi, and plants.

The term “comprising”, used in the claims, is “open ended” and means the elements recited, or their equivalent in structure or function, plus any other element or elements which are not recited. It should not be interpreted as being restricted to the means listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a composition comprising x and z” should not be limited to compositions consisting only of components x and z. Also, the scope of the expression “a method comprising the steps x and z” should not be limited to methods consisting only of these steps.

Unless otherwise indicated, all numbers used in this specification are to be understood as being modified in all instances by the term “about”. Unless specifically stated, as used herein, the term “about” is understood as within a range of normal tolerance in the art, for example within two standard deviations of the mean. In one embodiment, the term “about” means within 10% of the reported numerical value of the number with which it is being used, preferably within 5% of the reported numerical value. For example, the term “about” can be immediately understood as within 10%, 9%, 8%, 7%, 6%, 5%, 4%, 3%, 2%, 1%, 0.5%, 0.1%, 0.05%, or 0.01% of the stated value. In other embodiments, the term “about” can mean a higher tolerance of variation depending on for instance the experimental technique used. Said variations of a specified value are understood by the skilled person and are within the context of the present invention. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges, for example from 1-3, from 2-4, and from 3-5, as well as 1, 2, 3, 4, 5, or 6, individually. This same principle applies to ranges reciting only one numerical value as a minimum or a maximum.

Unless otherwise clear from context, all numerical values provided herein are modified by the term “about”. Other similar terms, such as “substantially”, “generally”, “up to” and the like are to be construed as modifying a term or value such that it is not an absolute. Such terms will be defined by the circumstances and the terms that they modify as those terms are understood by those of skilled in the art. This includes, at very least, the degree of expected experimental error, technical error and instrumental error for a given experiment, technique or an instrument used to measure a value.

As used herein, the term “and/or” includes any and all combinations of one or more of the associated listed items. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The invention will now be illustrated by the following non-limiting Examples.

### EXAMPLES

### List of Abbreviations

RPM—Revolutions per minute
RCF—Relative centrifugal force
CFU—Colony forming unit
PDBC—Potato dextrose broth with 20 μg/ml chloramphenicol
PDAC—Potato dextrose agar with 20 μg/ml chloramphenicol
PDAT—Potato dextrose agar with 12 μg/ml tetracycline
DMSO—Dimethyl sulfoxide
LB—LB broth
LBA—LB agar
SCH—Schmittner medium
2:PDBC—PDBC diluted 2 fold by sterile distilled water
PDA—Potato dextrose agar
PDBT—Potato dextrose broth with 12 μg/ml tetracycline

### Example 1. Microplate-Based Assay of 1-Phenyl-Tetralin Compounds Bioactivity Against Puccinia sorghi

Background: Puccinia sorghi is a fungus of belonging to the Basidiomycetes and it is an air borne pathogen. Puccinia spores were grown on corn plants in a growth room and fresh spore suspension is prepared from the infected corn leaves for each experiment. Since Puccinia sorghi is an obligatory pathogen and does not grow on synthetic medium, the germination of the spores was monitored as indication for 1-phenyl-tetralin compounds bioactivity.
Summary: Diluted in DMSO 1-phenyl-tetralin compounds (Compound 1: (5R,6R,7R)-5-(3,4-dihydroxyphenyl)-6,7-dimethyl-5,6,7,8-tetrahydronaphthalene-2,3-diol, Compound 2: 4-((1R,2R,3R)-7-hydroxy-6-methoxy-2,3-dimethyl-1,2,3,4-tetrahydronaphthalen-1-yl)benzene-1,2-diol, and Compound 3: (1S,4R)-4-(3,4-dichlorophenyl)-N-methyl-1,2,3,4-tetrahydronaphthalen-1-aminium chloride) were added separately to microplate wells and mixed with freshly prepared spore suspensions. The germination of the spores was monitored by visual inspection under the microscope.

The following materials, methods and equipment were used:

Materials: Tween® 20 (Tidea Company INC) non-ionic detergent, DMSO—dimethyl-sulfoxide (J. T. Baker—Poland) solvent, chloramphenicol (Alfa Aesar—UK)
Equipment: Centrifuge, Shaker, Incubator, Microscope, Filtration system

### Method:

### A. Puccinia Spore's Preparation

### Preparation of Corn Seedling for Inoculation:

1) Use 120×80×80 mm pots.
2) Use standard garden soil with fertilizer.
3) Use corn seeds of a sensitive variety.
4) Put the pots in a small tray.
5) Fill the pots with the soil to the top.
6) Make a small round grove for the seeds.
7) Plant about 10 seeds of corn in each pot.
8) Cover the seeds with additional soil.
9) Add water into the tray—about 100 ml for each pot. Soil should be wet, and no water should be left in the tray after 24 h.
10) Grow the corn for 7 days in growth room at 22° C. until the second leaf is emerged.
   B. Preparation of Spore Suspension [from Corn Leaves] for Inoculation and for Germination Study
1) Insert 20 corn leaves with spores into a sterile 50 ml tube.
2) Add 50 ml of ice cold 0.05% Tween® 20 solution.
3) Insert the tube into a sealed, ice cooled, plastic box.
4) Shake the box using a shaker at 300 RPM for 15 min.
5) Transfer the suspension (without the leaves) into a clean sterile 50 ml tube.
6) Keep the tube with the spore suspension on ice.

### C. Inoculation Preparation

1) Dilute the spore suspension to 300 ml using cold 0.05% Tween® 20 solution in water.
2) Check the spore concentration in the suspension—the concentration should be about 600 spores/ml and the suspension should have a light brown colour.
3) Keep the spore suspension on ice.

### D. Germination Assessment

1) Prepare filtration system with 5-micron membrane and wash the membrane with the sterile cold water.
2) Suspend and decant the spore suspension from the 50 ml tube slowly into the filtration system to the centre of the membrane—spores should accumulate on the membrane.
3) Wash the spores to discard bacteria and other fungi spores—stop the vacuum and spray cold sterile water to suspend and wash the spores, resume the vacuum.
4) Repeat spore wash twice more.
5) Insert the membrane with the spores into the tube with 30 ml sterile cold 0.05% Tween® 20 and shake the tube by hand.
6) Remove the membrane and discard it.
7) Decant filtered liquid to the sink and wash the filtration system with tap water and dry it.
8) Add 30 μl of Chloramphenicol stock solution (20 mg/ml) to spores' suspension up to final concentration of 20 μg/ml.
9) Filter the spore suspension through 8 layers of gauze into a 50 ml tube.
10) Check the spore concentration in the suspension—the concentration should be about 7.5×10³ spores/ml and should have a brown colour. Thirty ml of spore suspension should be enough for the screening of 20 microplates.
11) Keep the spore suspension on ice.

### E. Seedlings Infection

1) Transfer the spore suspension into 250 ml beaker.
2) Mix the spores' suspension with stirrer at 500 RPM to keep the spores suspended.
3) Dip the leaves of all the seedlings in the pot into the spore suspension for 10 min.
4) Put the pots with the inoculated seedlings in a moist chamber with heated water at 22° C. and 99% humidity for 24 h (heat the water to 32° C.).
5) After 24 h, transfer the pots to the growth room and cover the seedlings with the plastic bags
6) Grow the corn in growth room at 22° C.
7) After 7 days from inoculation, brown spots should be observed on the leaves.
8) After 11 days from inoculation remove the plastic bags to prevent fungal contamination and use a rubber band to hold the seedlings together.
9) After 12 days from inoculation leaves with spores can be collected for spore suspension preparation.

### F. Microplate Preparation for Bioactivity Screening Based on Puccinia Spore's Germination Assay

1) Take a plate with the stock solution of 1% 1-phenyl-tetralin compounds in DMSO solvent from the −20° C. freezer and thaw it on the bench for at least 20 min.
2) Take also a control plate with materials from the −20° C. freezer and thaw it on the bench for at least 20 min.
3) All microplates should contain 10 μl of stock 1-phenyl-tetralin compounds solution.
4) Add 15 μl of Puccinia spore suspension to all wells of microplates. Suspend the spores by the pipette (up and down) before transferring the spore suspension into the wells to obtain a final concentration of 25 ppm.
5) Seal all plates with transparent sealer.
6) Centrifugate all test plate at 1000 RCF for is and stop to collect the liquid at the bottom of the plate.
7) Shake all plates at 1000 RPM for 10 seconds and check for spore dispersion in the well.
8) Insert all plates to a plastic box and put the box in the incubator at 25° C. overnight.

### G. Screening of Plates

1) Screen the plate 12-24 h after suspension preparation using the microscope at 10×10 magnification.
2) Compare spore germination of each well to spore germination of the control plate wells (wells containing commercially available fungicides or 0.5% DMSO solution).
3) Report spore germination in Excel sheet:
   Type 1—if spores have germinated properly (normal tube elongation);
   Type 2—if spores have germinated poorly or not normal in any way (very short germination tubes, low frequency of germinating spores, damaged tubes);
   Type 3—if spores have not germinated at all (sound spores, no tubes).
4) Calculate the number of repeats of scores 2 or 3 for each material.
5) Calculate the sum of scores of 2 and 3 for each material.
6) Best score: number of repeats=4, sum of scores=12.

See results in Example 9.

### Example 2. Microplate-Based Assay of 1-Phenyl-Tetralin Compounds Bioactivity Against Rhizoctonia solani

Summary: Diluted in DMSO 1-phenyl-tetralin compounds (Compound 1, 2 or 3) were added separately to microplate wells and mixed with 50 μl of hyphae suspension and growth of the fungus, starting from blended hyphae, was monitored by plate reader and visual inspection.

The following Materials, methods and equipment were used:

### Materials: PDAC, PDBC, DMSO.

Equipment: Plate reader, Centrifuge, Shaker, Incubator.

### Method:

A. Inoculum Preparation of Rhizoctonia solani Hyphae
1) Grow Rhizoctonia on PDAC in 90 mm petri plates to get growing hyphae within 1-4 days.
2) Add 50 ml of PDBC medium into a sterile 250 ml Erlenmeyer flask.
3) Cut the solid medium by scalpel to several small pieces and insert them into the Erlenmeyer flask.
4) Grow the culture for 2-4 days using shaker at 27° C. and 150 RPM.
5) Discard the liquid and pour the hyphae on an empty Petri dish.
6) Cut many small pieces from the hyphae using a scalpel and insert them into a sterile 250 ml Erlenmeyer flask with 50 ml of PDBC medium.
7) Prepare 4 bottles with culture and grow for 3 days at 27° C. shaking at 150 RPM.
8) Chill the culture in the fridge for 1 h.
9) Pour the cold culture into a 250 ml beaker.
10) Add 20 ml of cold PDBC, so that the mixture will cover the blender knife.
11) Blend the culture with a blender for 2 min on ice at maximum speed, move the blender up and down several times.
12) Keep the mixture on ice.
13) Transfer about 5 ml of the blended mixture into a 15 ml tube on ice.
14) Homogenize the culture in the 15 ml tube for 2 min on ice, move the tube up and down as needed.
15) Homogenize several batches of 5 ml as above to prepare the amount that is needed (5 ml of homogenized culture would make about 100 ml of inoculum).
16) Dilute a portion of the homogenate 10-fold to check the concentration of the homogenate. The concentration of the suspension should be 4×10⁴ CFU/ml (diluted 10-fold concentration should be 4000 CFU/ml).
17) Dilute the inoculum stock 1:20 in PDBC—1 ml in 20 ml, or calculate the dilution needed, to prepare final concentration of 2000 CFU/ml. The amount in each well should be about 100 CFU.

### B. Microplate Preparation for 1-Phenyltetralin Compounds Bioactivity Experiment

1) Take a stock solution of one of the purified 1% 1-phenyl-tetralin compounds in DMSO from the −20° C. freezer and thaw it on the bench.
2) Take 1 μl of stock solution of 1% 1-phenyl-tetralin compounds and dilute up to 250 ppm with 39 μl of water.
3) Take 10 μl of the diluted (250 ppm) 1-phenyl-tetralin compounds solution into the wells of the microplate using a multi-pipette.
4) Add 40 μl of vigorously mixed spore suspension inoculum to the wells of the microplate using a multi-pipette.
5) Shake the plate for 10 min at 2000 RPM to mix the 1-phenyl-tetralin compounds with the hyphae suspension.
6) Centrifugate the plate at 1000 RCF for is and stop to collect the liquid at the bottom of the plate.
7) Keep the microplate on the bench until it is read by the plate reader.
8) Read the plate using the plate reader.
9) Collect the plates on the bench.
10) Insert collected plates to a plastic box with cloth cover and put the box in the incubator at 27° C.

### C. Screening of Plates

1) Screen plate at 3 more dates: 3 d, 7 d, 14 d and 21 d following the assay start.
2) Calculate the difference of absorbance between each screen and the read at zero time.
3) Calculate the percentage of growth inhibition of each well at each time point. Use the results of the DMSO treatment of the control plate as 100% growth.

See results in Example 9.

### Example 3. Microplate-Based Screening of 1-Phenyl-Teralin Compounds with Potential Bioactivity Against Pythium aphanidermatum

Summary: Diluted in DMSO 1-phenyl-tetralin compounds (Compound 1, 2 or 3) were added separately to microplate wells and mixed with 50 μl of zoospores in PDBC suspension and the growth of the fungus, starting from zoospores, was monitored by plate reader and visual inspection.

The following Materials, methods and equipment were used:

### Materials: SCH, PDBC, DMSO.

Equipment: Plate reader, Centrifuge, Shaker, Incubator.

### Method:

### A. Inoculum Preparation of Pythium Hyphae

1) Grow Pythium aphanidermatum on SCH in 90 mm petri plates to get sporulating hyphae. Each plate will produce 50 ml of zoospores suspension which will be enough for bioactivity screening for ten 96-well plates.
2) Add 60 ml of sterile water into a sterile 250 ml Erlenmeyer flask.
3) Cut the solid medium of 2 plates by scalpel to 12 pieces (each plate) and insert them into the Erlenmeyer flask (the solid pieces should be covered by the water).
4) Let the hyphae sporulate overnight at 17° C.
5) Shake the Erlenmeyer flask by hand to suspend the zoospores.
6) Filter the suspension into 50 ml tube through 16-layer gauze.
7) Transfer the suspension into a sterile 500 ml bottle.
8) Discard the solids and disinfect the Erlenmeyer flask with hypochlorite.
9) Chill the zoospore suspension on ice.
10) Evaluate the zoospores concentration in the suspension (the concentration should be 1000-4000 spores/ml).
11) Dilute the suspension by sterile fridge cold distilled water in a sterile 500 ml bottle.
12) Add the same volume (as the suspension) sterile fridge cold 2:PDBC to get 500-2000 spores/ml inoculum. This dilution will result in the amount of 25-100 zoospores in each well.
13) Keep the zoospore suspension inoculum on ice.

### B. Microplate Preparation for 1-Phenyl-Tetralin Compounds Bioactivity Experiment

1) Take a stock solution of purified 1% 1-phenyl-tetralin compounds (1, 2 or 3) in DMSO and thaw it on the bench for at least 20 min.
2) Take 1 μl of stock solution of 1% 1-phenyl-tetralin compounds and dilute up to 250 ppm with 39 μl of water.
3) Take 10 μl of the diluted (250 ppm) 1-phenyl-tetralin compounds solution into the wells of the microplate using a multi-pipette.
4) Add 40 μl of zoospore suspension inoculum to the wells of the microplate using a multi-pipette. Mix the spore suspension vigorously by hand and decant amount needed for one plate (5 ml) to keep the zoospores well suspended.
5) Seal the plate with transparent sealer.
6) Shake the plate for 10 min at 2000 RPM to mix the 1-phenyl-tetralin compounds with the hyphae suspension.
7) Centrifugate the plate at 1000 RCF for 1 second and stop to collect the liquid at the bottom of the plate.
8) Keep the microplate on the bench until it is read by the plate reader.
9) Read the plate using the plate reader.
10) Collect the plates on the bench.
11) Insert collected plates to a plastic box with cloth cover and put the box in the incubator at 27° C.
   C. Screening of plates
1) Read out the plate at 3 more dates: 3 d, 7 d, 14 d and 21 d following the assay start.
2) Calculate the difference of absorbance between each readout and the readout at zero time.
3) Calculate the percentage of growth inhibition of each well at each time point. Use the results of the DMSO treatment of the control plate as 100% growth.

See results in Example 9.

### Example 4. Microplate-Based Screening of 1-Phenyl-Tetralin Compounds with Potential Bioactivity Against Botrytis cinerea

Summary: Microplates with Compound 1, 2 or 3 diluted in DMSO was mixed with frozen spore suspension and the growth of the fungus was monitored, starting from frozen spores by visual inspection.
Background: Botrytis cinerea is a fungus belonging to Ascomycetes and it is an air-borne pathogen. It is quite easy to produce large amounts of Botrytis spores, which survive in liquid 60% glycerol at −20° C. Thus, we used frozen spores' stocks in the bioactivity screening experiments rather than prepare fresh spores for each experiment.
Aim: To determine the effect of a 1-phenyl-tetralin compound on the survival and growth of Botrytis.

The following Materials, methods and equipment were used:

### Materials: PDAC, PDBC, DMSO.

Equipment: Centrifuge—Eppendorf 5810R; Shaker—Scientific Industries, Multi Microplate Genie; Incubator—Pol-Eco Aparatura; Plate reader.

### Method:

### A. Botrytis Spore Suspension Preparation

1) Put a PDAC block of Botrytis in the middle of a PDAC plate and grow 12 days at 22° C.
2) Chill the plate in the fridge for at least 1 h.
3) Add 25 ml of fridge cold, sterile, 60% glycerol solution to the tube.
4) Cut the agar with the hyphae and spores to 8 pieces and insert them into a 50 ml sterile tube.
5) Shake for 1 min at 3000 RPM.
6) Keep spores on ice during the whole process.
7) Transfer the liquid to a new 50 ml sterile tube—about 25 ml should be recovered.
8) Filter the spore suspension through 16 layer of gauze cloth directly into a clean sterile 50 ml tube to discard the hyphae: about 20 ml should be recovered.
9) Calculate the spore concentration and dilute by cold sterile 60% glycerol solution to get 2×10⁵ spores/ml.
10) Dispense 1 ml aliquot of spore suspension into 1.5-ml tubes—each aliquot should be sufficient for 20 plates for screening.
11) Store the spore suspension at −20° C.

### B. Spore Suspension Preparation for Screening

1) Take 200 μl frozen spore suspension from the freezer and thaw it on ice.
2) Mix the spore suspension with 20 ml ice cold PDBC in a 50-ml tube, to make 2×10⁵ spores per ml concentration for 4 microplates.
3) Use this suspension for screening experiments.

### C. Sterilize the Following Items Using the Autoclave:

50-ml tubes×36; Reservoir×4; 400-ml PDBC.

### D. Microplate Preparation for 1-Phenyl-Tetralin Compounds Bioactivity Experiment

1) Take a stock solution of purified 1% 1-phenyl-tetralin compound in DMSO and thaw it on the bench for at least 20 min
2) Take 1 μl of stock solution of 1% 1-phenyl-tetralin compound and dilute up to 250 ppm with 39 μl of water.
3) Take 10 μl of the diluted (250 ppm) 1-phenyl-tetralin compound solution into the wells of the microplate using a multi-pipette.
4) Add 40 μl of spore suspension inoculum to the wells of the microplate, mix the spore suspension vigorously by hand and decant amount needed for one plate (5 ml), to keep the spores well suspended.
5) Seal the plate with transparent sealer.
6) Shake the plate for 10 min at 2000 RPM to mix the materials with the hyphae suspension.
7) Centrifugate the plate at 1000 RCF for 1 second and stop, to collect the liquid at the bottom of the plate.
8) Keep the microplate on the bench until it is read by the plate reader.
9) Evaluate the fungal growth the plate using the plate reader and visual inspection.
10) Collect the plates on the bench.
11) Insert collected plates to a plastic box, with cloth cover and put the box in the incubator at 22° C.

### E. Readout of the Plates

1) Collect the plate readouts at 3 more dates: 3 d, 7 d, 14 d and 21 d following the assay start.
2) Calculate the difference of absorbance between each readout and the readout at zero time.
3) Calculate the percentage of growth inhibition of each well at each time span, use the results of the DMSO treatment of the control plate as 100% growth.
4) “Hits” are those compounds which have 4 repeats with “clear well” or displayed less than 20% pathogen growth compared to DMSO 0.5% solution.

See results in Example 9.

### Example 5. Microplate-Based Screening of 1-Phenyl-Tetralin Compounds with Potential Bioactivity Against Fusarium oxysporum

Summary: Compound 1, 2 or 3 diluted in DMSO was added to microplate wells and mixed with freshly prepared spore suspension and growth of the fungus, starting from frozen spores, was monitored using the plate reader and by visual inspection.
Background: Fusarium is a fungus of belonging to the Ascomycetes, and it is a soil borne pathogen. It is quite easy to produce large amounts of spores of Fusarium and they survive in liquid 60% glycerol at −20° C. Thus, we used frozen spores' stock in the bioactivity screening experiments rather than prepare fresh spores for each experiment.
Aim: To determine the effect of 1-phenyl-tetralin compounds on the survival and growth of Fusarium.

The following materials, methods and equipment were used:

### Materials: PDAC, PDBC, DMSO.

Equipment: Plate reader, Centrifuge, Shaker, Incubator.

### Method:

### A. Fusarium Spore Suspension Preparation

1) Put agar block of growing Fusarium on PDAC in the middle of a PDAC plate and grow for 9 days at 25° C.
2) Chill the plate in the fridge for at least 1 h.
3) Add 30 ml of fridge cold, sterile, 60% glycerol solution to the 50-ml tube.
4) Cut the agar with the hyphae and spores, from one plate, to small pieces, by scalpel, and insert them into the 50 ml-tube with 30 ml 60% glycerol.
5) Shake for 1 min at 3000 RPM.
6) Keep spores on ice during the whole process.
7) Transfer the liquid to a new 50 ml sterile tube—about 25 ml should be recovered.
8) Filter the spore suspension through 16 layer of gauze cloth directly into a clean sterile 50 ml tube to discard the hyphae.
9) Calculate the spore concentration (at 40×10 magnification) and dilute by cold sterile 60% glycerol solution to get 2×10⁵ spores/ml.
10) Aliquot 1 ml of spore suspension into 1.5-ml tubes—each aliquot should yield 20 plates for screening.
11) Store the spore suspension at −20° C.

### B. Spore Suspension Preparation for Screening

1) Take 1 ml frozen spore suspension from the freezer and thaw it on ice.
2) Mix 200 μl spore suspension with 20 ml fridge cold PDBC in a 50-ml tube to make 2000 spores/ml suspension.
3) Use this amount to screening of 4 microplates with 100 spores per well.

### C. Microplate Preparation for 1-Phenyl-Tetralin Compounds Bioactivity Experiment

1) Take a stock solution of purified 1% 1-phenyl-tetralin compound in DMSO and thaw it on the bench for at least 20 min.
2) Take 1 μl of stock solution of the 1% 1-phenyl-tetralin compound and dilute up to 250 ppm with 39 μl of water.
3) Take 10 μl of the diluted (250 ppm) 1-phenyl-tetralin compounds solution into the wells of the microplate using a multi-pipette.
4) Add 40 μl of spore suspension inoculum to the wells of the microplate using a multi-pipette.
5) Mix the spore suspension vigorously by hand and decant amount needed for one plate (5 ml) to keep the spores well suspended
6) Seal the plate with transparent sealer
7) Shake the plate for 10 min at 2000 RPM to mix the materials with the hyphae suspension
8) Centrifugate the plate at 1000 RCF for is and stop to collect the liquid at the bottom of the plate
9) Keep the microplate on the bench until it is read by the plate reader
10) Read the plate using the plate reader
11) Collect the plates on the bench
12) Insert collected plates to a plastic box with cloth cover and put the box in the incubator at 25° C.

### D. Readout of the Plates

Collect the readout of the plate at 3 more dates: 3 d, 7 d, 14 d and 21 d following the assay start
1) Calculate the difference of absorbance between each readout and the readout at zero time
2) Calculate the percentage of growth inhibition of each well at each time course. Use the results of the DMSO treatment, of the control plate as 100% growth.

See results in Example 9.

### Example 6. Microplate-Based Screening of 1-Phenyl-Tetralin Compounds with Potential Bioactivity Against Phytophthora infestans

Background: Phytophthora infestans is an obligatory pathogen from Oomycetes which is very difficult to grow on synthetic medium. Therefore, the bioactivity screening system based on leaf discs prepared from detached tomato leaves were used.
Summary: Compounds 1, 2 or 3 diluted in DMSO were added to tomato leaf discs infected with Phytophthora and the disease progress was monitored by visual inspection.
General description: Inoculation and maintenance on tomato leaves, preparation of spore suspension, their growth on leaf discs in microplates and inspection by magnifying glass of Phytophthora infestans severity of infection.

The following materials, methods and equipment were used:

### Method:

### A. Preparation of Tomato Seedling for Leaves Production for Inoculation

1) Use seedling pots of size 120×80×80.
2) Use standard garden earth with fertilizer.
3) Use 4 weeks old tomato seedlings.
4) Put 6 pots in a small tray.
5) Put one seedling in each pot.
6) Add water into the tray—about 100 ml for each pot. Earth should be wet, and no water should be left in the trey after 24 h.
7) Grow the tomato plants in growth room at 22° C. and 16 h light/darkness conditions.
8) When plants are grown (4 weeks after planting) transfer them to a 5 L pot and fertilize every week with.

### B. Preparation of Tomato Leaves for Inoculation

1) Put two pieces of sterile paper in a square Petri dish.
2) Work in sterile conditions.
3) Use leaves of 5 weeks old tomato plants or older.
4) Cut the leaves by a sterile scalpel.
5) Add 20 ml sterile distilled water to wet the paper (the paper should be maximally wet, but without additional dripping water).
6) Put about 6 lobes of leaves in a square Petri dish, on the wet paper (with the lower side of the leaves up).
7) Cover the plate with its lid.

### C. Preparation of Inoculum and Leaf Discs Infection

### Preparation of Sporangium Suspension

1) Put 10 lobes of tomato leaf infected with Phytophthora (4-6 days after infection), in a sterile 50 ml tube, fill in 40 ml of fridge cold, sterile, distilled water.
2) Mix the tube gently by hand, to transfer sporangium into the water, but avoid disintegration of the leaf.
3) Filter the spore suspension through 16 layers of gauze into a 50-ml tube.
4) Calculate the spore concentration—use microscope with 200× magnification. A concentration of 6000 sporangium/ml is expected.
5) Chill the tube on ice.

### D. Sporangium Wash and Concentration by Filtration

1) Prepare filtration system with membrane (0.65 micron-5 micron), and wash the membrane with sterile cold water.
2) Suspend and decant the spore suspension from the 50 ml tube slowly, into the filtration system. Use low vacuum, don't let the membrane dry—leave 4 ml unfiltered suspension on the filter.
3) Wash the spores to discard bacteria and other fungi spores (use 40 ml water to wash)—spray cold sterile water to suspend and wash the spores.
4) Repeat spore wash 5 more times. Do not let the membrane dry. Leave 4 ml unfiltered suspension.
5) Collect the spore suspension using a 1000 μl pipettor into a clean 50-ml tube.
6) Insert the membrane into the 50-ml tube mix gently to suspend the sporangium, which stick to the membrane.
7) Discard the membrane to be autoclaved.
8) Discard liquid, and disinfect the filtration system using hypochlorite for 30 min.
9) Wash the filtration system with tap water and dry the filtration system on a paper in a plastic basket.
10) Calculate the sporangium concentration—use microscope with 200× magnification—10,000-50,000 sporangium/ml concentration is expected.
11) Keep the sporangium suspension on ice.

### E. Inoculation of Spores on Detached Leaves for Maintenance of Phytophthora

1) Spray 1000 μl of Phytophthora spore suspension on all the lobes of leaves in one square dish and cover the dish.
2) Infect the leaves with the fungus on and keep the leaves into the incubator at 17° C. in the dark for 24 h.
3) Transfer the plates for additional 3-5 days into the incubator at 22° C., with 12 h light, for sporangium growth.

### F. Tomato Leaf Discs Microplate Preparation for Screening

1) Take the 48 wells plate.
2) Prepare sterile water agar 0.5%, use it preheated, but cold.
3) Add 100 μl sterile water agar 0.5% to the microplate wells.
4) Put tomato leaf discs prepared from 3^{rd}, 4^{th} or 5^{th} leaf, into microplate wells. Press the discs gently to ensure maximal contact with the liquid agar solution.

### G. Inoculation of Spores on Leaf Discs Microplate for Materials Screening

1) Insert the spore suspension into the microplate for testing.
2) Seal the chemicals microplate with transparent sealer.
3) Shake the microplate for 10 min at 2000 RPM to mix the materials with the added spore
suspension.
4) Centrifugate the microplate at 1000 RCF for is and stop to collect the liquid at the bottom of the plate.
5) Add 5 μl spore suspension onto the middle of each disc of the microplate.
6) Seal the leaf discs plate with transparent sealer.
7) Insert the sealed leaf discs plates into the incubator at 17° C. for 24 h in the dark and then at 22° C., with 12 h light/darkness regime for 3-5 days.
8) Perform the bioactivity evaluation.

### H. Bioactivity Evaluation

1) Screen plate at one time point: 5 days after suspension preparation using a ×5 magnification glass.
2) Report infected discs in Excel sheet:
   Type 1—if discs are fully infected;
   Type 2—inconclusive;
   Type 3—if discs are not infected at all.
3) Calculate the number of repeats of scores of 3 for each material.
4) Calculate the sum of scores of 3 for each material.
5) Best score was calculated as following: number of repeats=4, sum of scores=12.
6) “Hits” are those materials which have 4 or 3 repeats, and sum of 6-12.

See results in Example 9.

### Example 7. Microplate-Based Screening of 1-Phenyl-Tetralin Compounds for Potential Bioactivity Against Pseudomonas syringae

Background: Pseudomonas is a rod-shaped Gram-negative bacterium. Frozen bacterial stock in 60% glycerol was used as an inoculum for the bioactivity screening experiments.
Summary: Compounds 1 or 2 diluted in DMSO were added to microplate wells and mixed with frozen bacteria suspension and growth of the Pseudomonas was monitored by visual inspection. The following materials, methods and equipment were used:

### Materials: LB, LBA, DMSO.

### Equipment: Centrifuge, Shaker, Incubator.

### Method:

### A. Pseudomonas Suspension Preparation:

1) Grow Pseudomonas on LBA plates at 28° C. for 2 days to get a single colony.
2) Transfer a single colony using a sterile toothpick into a 50 ml sterile tube containing 5 ml LB and grow for 24 hours at 28° C. and 150 RPM.
3) Chill the tube in the fridge for 1 h.
4) Add 7.5 ml of fridge cold, sterile, glycerol solution to the tube—to get 60% glycerol solution.
5) Mix well but gently to get perfect mixing—use vortex at 1000 RPM.
6) Aliquot 100 μl of bacteria suspension in 60% glycerol into 1.5-ml tubes—each aliquot should be enough for screening of 10 microplates.
7) Store the bacteria suspension in 60% glycerol at −20° C.

### B. Pseudomonas Suspension Preparation for Bioactivity Screening Experiment:

1) Take 1.5-ml tube with 100 μl frozen Pseudomonas suspension from the freezer and thaw it on ice.
2) Prepare in the hood 50-ml tubes with 40 ml fridge cold LB.
3) Mix 40 μl of bacteria suspension with 40 ml fridge cold LB in a 50-ml tube. This amount is enough for activity screening of 10 microplates.
4) Use this suspension for bioactivity screening experiments.

### C. Microplates' Preparation for Bioactivity Screening Experiment:

1) Take a stock solution of purified 1% 1-phenyl-tetralin Compound 1 or 2 in DMSO and thaw it on the bench for at least 20 min.
2) Take 1 μl of stock solution of the 1% 1-phenyl-tetralin compound and dilute up to 250 ppm with 39 μl of water.
3) Take 10 μl of the diluted (250 ppm) 1-phenyl-tetralin compound solution into the wells of the microplate using a multi-pipette.
4) Add 80 μl of bacteria suspension with growth medium to each well of the microplate using a multi-pipette.
5) Seal the plate with transparent sealer.
6) Shake the plate for 10 min at 2000 RPM to mix the 1-phenyl-tetralin compound with the bacteria suspension.
7) Centrifugate the plate at 1000 RCF for 1 second and stop to collect the liquid at the bottom of the plate.
8) Insert the plates to a plastic box with cover and put the box in the incubator at 28° C.

### D. Bioactivity Screening of Microplates:

1) Screen the microplate at 5 dates: 3, 5, 7, 14 and 21 days after inoculation.
2) Use a lamp to visually evaluate the bacterial growth.
3) Prepare plates for screening: shake plate at 2000 RPM for 2 min to suspend the bacteria and then centrifuge plate at 1000 RCF for a few seconds.
4) Screen the microplates after removing their cover, if there is liquid on the cover (from inside) evaporate the liquid using a heated block at 60° C.
5) Compare the transparency of each well to the transparency of the control wells (wells containing control bactericide or 0.5% DMSO solution).
6) Record the results using the following interpretation: clear=3 (no growth of bacteria), turbid=1 (normal bacterial growth), inconclusive=2 (very low turbidity compared to growth in 0.5% DMSO solution).

See results in Example 9.

### Example 8. Microplate-Based Testing of 1-Phenyl-Tetralin Compounds for Potential Bioactivity Against Alternaria alternata

Background: Alternaria alternata is a major plant pathogen and cause large damage to many agricultural crops. Alternaria alternata is a fungus of belonging to the Ascomycetes, and it is an air borne pathogen. It is quite easy to produce large amounts of spores of Alternaria alternata, and they survive in liquid 60% glycerol at −20° C., that led to decision to use frozen spore stock in this screening rather than prepare fresh spores for each experiment.
Summary: Compound 1, 2 or 3 diluted in DMSO was added to microplate wells and mixed with frozen bacteria suspension and growth of Alternaria was monitored by visual inspection.

The following materials, methods and equipment were used:

### Materials: LB, LBA, DMSO.

### Equipment: Centrifuge, Shaker, Incubator.

### Method:

### A. Alternaria Spore Suspension Preparation

1) Put a PDAT block of Alternaria in the middle of a PDAT plate and grow for 21 days at 25° C. in a box with silica gel.
2) Chill the plate in the fridge for at least 1 h.
3) Add 25 ml of fridge cold, sterile water.
4) Cut the agar with the hyphae and spores from one plate to 8 pieces by scalpel and insert them into a 50-ml sterile tube.
5) Shake for 1 min at 3000 RPM.
6) Keep spores on ice during the whole process.
7) Transfer the liquid to a new 50-ml sterile tube—about 25 ml should be recovered.
8) Filter the spore suspension through 16-layers gauze cloth directly into a clean sterile 50 ml tube to discard the hyphae about 20 ml should be recovered.
9) The expected concentration of spores is 25,000 spore/ml.
10) Centrifugate the 50-ml tubes with the spores at 4500 RCF for 5 min. A small dark pellet should be seen at the bottom of the tube.
11) Discard the liquid gently and keep the spore's pellet with about 3 ml liquid in each tube.
12) Keep on ice.
13) Suspend the spores by vortex.
14) Collect the suspension from all tubes into one 50-ml tube.
15) Calculate the spore concentration (count×10 dilution at 20×10 magnification).
16) Centrifugate the 50-ml tube with the spores at 4500 RCF for 5 min. A small dark pellet should be seen at the bottom of the tube.
17) Adjust the spore concentration after the centrifugation (use the volumes ratio for the calculation). The concentration should be 5×10⁵, adjust the spore concentration by addition of water, or by reducing the amount of water in the tube with the spore pellet.
18) Add cold sterile glycerol (100%) to get 60% glycerol solution. Final spore concentration should be 2×10⁵.
19) Mix the spore suspension well.
20) Distribute aliquots of 1 ml of spore suspension into 1.5-ml tubes.
21) Store the spore suspension at −20° C.

### B. Spore Suspension Preparation for Screening

1) Take 400 μl frozen spore suspension from the freezer and thaw it on ice.
2) Mix the spore suspension with 20 ml ice-cold PDBC in a 50 ml tube, to make 2×10³ spores per 1 ml concentration for 4 microplates.
3) Use this suspension for screening experiments.

### C. Steam-Sterilize the Following Items Using the Autoclave:

50-ml tubes×36; Reservoir×4; 400-ml PDBC.

### D. Microplate Preparation for Screening Experiment

1) Take a stock solution of purified 1% 1-phenyl-tetralin compounds in DMSO from the −20° C. freezer and thaw it on the bench.
2) Take 1 μl of stock solution of 1% 1-phenyl-tetralin compounds and dilute up to 250 ppm with 39 μl of water.
3) Take 10 μl of the diluted (250 ppm) 1-phenyl-tetralin compounds solution into the wells of the microplate using a multi-pipette.
4) Add 40 μl of vigorously mixed spore suspension inoculum to the wells of the microplate using a multi-pipette and seal the plate with transparent sealer.
5) Shake the plate for 10 min at 2000 RPM to mix the materials with the hyphae suspension.
6) Centrifugate the plate at 1000 RCF for 1 second and stop, to collect the liquid at the bottom of the plate.
7) Collect the plates on the bench until all the plates are ready for incubation.
8) Insert collected plates to a plastic box and put the box in the incubator at 25° C.

### E. Screening of Plates

1) Screen plate at 3 dates: 7, 14 and 21 days after inoculation.
2) Use a lamp for visual assessment of compounds effect on fungal growth overtime.
3) Screen plates after removing their cover, if there is liquid on the cover (from inside) evaporate the liquid by a headed block at 60° C.
4) Compare the hyphal growth of each well to the hyphal growth of the control plate wells (wells containing commercially available fungicides or 0.5% DMSO solution).
5) The results were interpreted using the following grades: clear=3 (no growth of hyphae), normal hyphal structure=0 (normal growth), inconclusive=2 (solid structure of unexpected type, or partial cover of the area).

See results in Example 9.

### Example 9. Results of In Vitro Experiments Based on Protocols of Examples 1-8

### In-Vitro Screening Matrix

1-phenyl-tetralin compounds were screened against selected agricultural pests (as indicated in the tables below). The bioactivity values are in % and reflect the potential of eradicating the target pests.

Rules for Bioactivity Relative Value Calculation (Expressed in % from Maximal Value)
a. Puccinia sorghi, Phytophthora infestans—activity grade (1/2/3) X repeats #/12 (maximal value 3×4=12)×100
b. Alternaria alternata, Botrytis cinerea, Rhizoctonia solani, Sclerotinia sclerotiorum, Fusarium oxysporum, Pythium aphanidermatum—activity grade (1/2/3) X repeats #X days of activity/252 (maximal value 3×4×21=252)×100
c. Pseudomonas syringae, Pectobacterium caratovorum—activity grade (1/2/3) X repeats # X days of activity/168 (maximal value 3×4×14=168)×100

TABLE 1
Bioactivity values of Compound 1 on various target pests

| Pathogen | Relative Activity value (%) |
|---|---|
| Puccinia sorghi | 50 |
| Phytophthora infestans | 25 |
| Rhizoctonia solani | 50 |
| Pythium aphanidermatum | 100 |
| Pseudomonas syringae | 14 |

TABLE 2
Bioactivity values of Compound 2 on various target pests

| Pathogen | Relative Activity value (%) |
|---|---|
| Puccinia sorghi | 50 |
| Rhizoctonia solani | 14 |
| Pythium aphanidermatum | 20 |
| Pseudomonas syringae | 14 |

TABLE 3
Bioactivity values of Compound 3 on various target pests

| Pathogen | Relative Activity value (%) |
|---|---|
| Puccinia sorghi | 100 |
| Phytophthora infestans | 100 |
| Botrytis cinerea | 100 |
| Alternaria alternata | 100 |
| Rhizoctonia solani | 25 |
| Pythium aphanidermatum | 10 |
| Fusarium oxysporum | 16 |

In summary, 1-phenyl-tetralin compounds are demonstrated to be effective pesticides against the following pests: Puccinia sorghi (positive results are provided in in-planta results section below), Phytophthora infestans (positive results in tomato detached leaves validation experiments and greenhouse in-vivo validation experiments provided), Rhizoctonia solani, Pythium aphanidermatum, Alternaria alternata, Botrytis cinerea (positive results in in-vivo tomato validation experiments under greenhouse conditions provided), Fusarium oxysporum and Pseudomonas syringae.

### Statistical Analysis Used for Validation Experiments

To evaluate the effect of a tested compounds in infected plants compared to control plants (infected but not treated) the data was analysed by Student's t-test and the p-value is calculated. The minimum number of repeats in each experiment was 3. Results were considered significant if p<0.05. The data presented as mean with standard error mean from biological replicates. * means that p-value <0.05, ** means that p-value is <0.01, *** means that p-value is <0.001, # means that p-value <0.1, n.s.—means non-significant effect vs. control.

### Formulations Recipes Used for Validation Experiments

### Preparation of Formulation 1

Three types of stock solutions were used for final 1-phenyl-tetralin compound formulation preparation at 400 ppm (Formulation 1):

(A) 1-phenyl-tetralin compound solution in water and acetic acid.
A 1-phenyl-tetralin compound was dissolved in water to obtain 0.2% solution in water, followed by addition of 2% acetic acid. The final solution was sonicated for 5 mins at room temperature. The solution should be clear and colourless.
   (B) 0.4% Xanthan Gum in water (w/w).
   (C) 0.6% Silwet® in water (w/w). The final formulation which was applied to corn plants is composed of: 20% of stock solution A, 10% of stock solutions B and C, and 60% of water.

The final formulated 1-phenyl-tetralin compound was applied as 400 ppm or diluted to the required concentrations and applied to plants.

### Preparation of Formulation 2

Three types of stock solutions were used for final 1-phenyl-tetralin compound formulation preparation at 400 ppm:

(A) 1-phenyl-tetralin compound suspension in water.
A 1-phenyl-tetralin compound was grinded using grinder, and the grinded compound was used to obtain 1% suspension of the compound in sterile water. The 1-phenyl-tetralin compound original weight should be 50 mg. The 1-phenyl-tetralin compound was grinded in volume of 1 ml (50 oscillations/s, for 1 minute), repeated 5 times, to obtain the final volume of 5 ml suspension and final concentration of 1%.
   (B) 0.4% Xanthan Gum in water (w/w).
   (C) 0.6% Silwet® in water (w/w).

The final formulation which was applied to wheat plants is composed of:

4% of stock solution (A), 10% of stock solutions (B), 3.3% of stock (C), and 82.7% of water.

The final formulated 1-phenyl-tetralin compound was applied as 400 ppm or diluted to the required concentrations and applied to plants.

### Example 10. In Planta Validation in Corn

Protocol name: Puccinia sorghi infection of corn seedlings test
General description: Inoculation on corn, collection, Puccinia sorghi spores' suspension preparation and 1-phenyl-tetralin compounds 1 or 3 bioactivity evaluation against Puccinia sorghi.

The following materials, methods and equipment were used:

### Method:

### A. Preparation of Corn Seedlings for Inoculation

1) Use: 120×80×80 mm pots, standard garden soil with fertilizer and corn seeds of a rust sensitive variety.
2) Put pots in a tray and fill the pots with the soil to the top.
3) Make a small grove for the seeds.
4) Plant about 10 seeds of corn in each pot.
5) Cover the seeds with additional soil.
6) Add water into the tray—about 100 ml for each pot (fill the tray 3 times).
7) Grow the corn for 8 days in growth room at 22° C. (until the second leaf is emerged).
   B. Preparation of Spore Suspension [from Corn Leaves] for Inoculation
1) Insert 20 infected corn leaves with spores into a sterile 50-ml tube.
2) Add 50 ml of cold 0.05% Tween® 20 solution.
3) Insert the tube into a sealed, ice cold plastic box.
4) Shake the box using a shaker for 15 min at 3000 RPM.
5) Transfer the suspension (without the leaves) into a clean sterile 50-ml tube.
6) Filter the spore suspension through 16 layers of gauze into another sterile 50-ml tube.
7) Keep the tube with the spore suspension on ice.
8) Wash and concentrate the spores on a 5-micron membrane filter. Wash 4 times with ice-cold sterile water and collect the spores in 0.05% Tween® 20 solution.
9) For inoculation, dilute the spore suspension to get spore concentration of 8000 spores/ml using cold 0.05% Tween® 20 solution.

### C. Growth Room Experiments

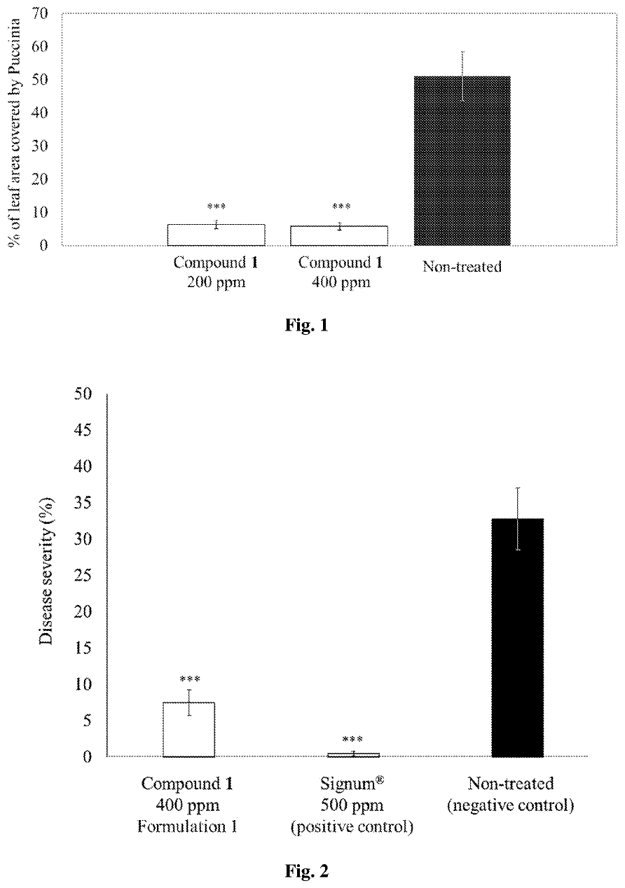

1) Use 8 days old seedlings prepared as explained above.
2) Prepare treatment for spraying—about 1 ml for each plant.
3) Add Tween® 20 up to 0.05% to the treatments.
4) Spray treatment on to the leaves till full saturation (use a spray bottle) and let dry in the growth room.
5) Repeat spray on the second day and let dry.
6) On the second day (after about 4 hours from the treatment spray) inoculate plants using the Puccinia spore suspension.
7) Use spraying bottle to spray about 1 ml (till full saturation) of the spore suspension on to the leaves of 9 days old corn seedlings.
8) Put the pots with the inoculated seedlings in a dark moist chamber with heated water at the bottom. The chamber should be held in a room at 22° C. for 24 h with 99% humidity (the temperature of the heated water should be 32° C.).
9) After 24 h transfer the pots to the growth room.
10) Grow the corn in the growth room at 22° C.
11) After 7 days from inoculation, brown spots should be seen on the leaves.
12) Record leaf coverage of Puccinia brown spots after 9 days from inoculation.
13) Compare leaf coverage of treated seedlings to water treated seedlings.
   Compound 1 Formulation for Exp. 343 (with Reference to FIG. 1)

Compound 1 was dissolved in dimethyl-sulfoxide solvent with 1:9 weight to weight ratio and then brought up to the final volume used for the validation with double distilled water. Before spraying, the non-ionic detergent Tween® 20 was added to final concentration of 0.05%.

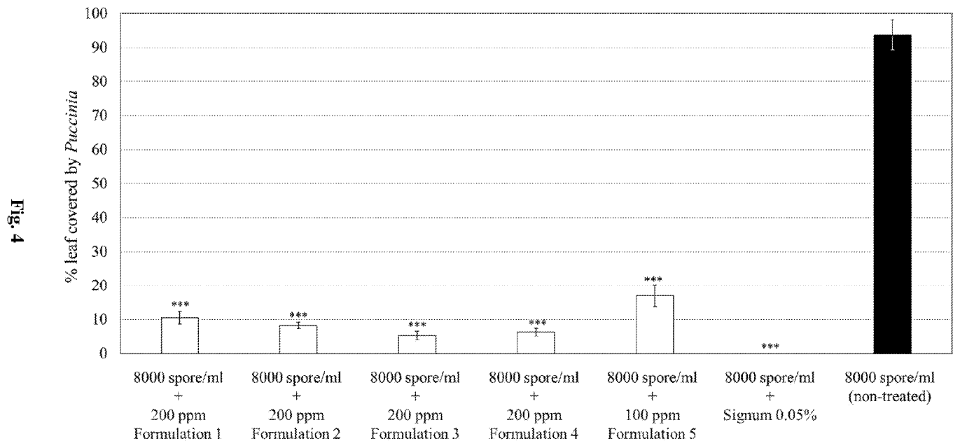

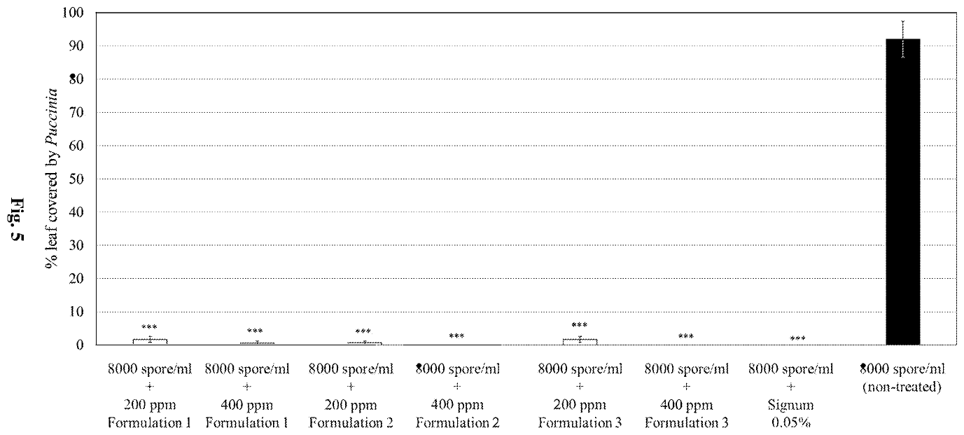

Compound 3 Formulations 1-5 for Exps. 270, 284, 294 (with Reference to FIGS. 4-6)

Compound 3 was dissolved in absolute ethanol with 1:36 weight to weight ratio or in dimethyl-sulfoxide with 1:17 weight to weight ratio, sonicated for 5 mins and then another part of non-ionic detergent either Tween® 20 with weight to weight 1:4.5 ratio to Compound 3 or Silwet® in 1:1 weight to weight ratio to Compound 3 was added for formulation finalisation. In some cases, Na₂CO₃ was used to adjust pH to 6.

### Results

Several experiments were conducted under controlled environment in growth rooms where the potential of Compound 1 and Compound 3 to prevent and control Puccinia sorghi in corn plants was estimated (FIGS. 1, 4, 5 and 6). Compound 1 and Compound 3 performed very well and showed very good efficacy under controlled growth conditions. The average efficacy of the 1-phenyl-tetralin compounds in preventing and controlling the Puccinia sorghi was 95.17% at 200 ppm and 97.06% at 400 ppm.

### Example 11. Validation in Planta Experiments in Wheat Infected with Leaf Rust (Puccinia triticina) Under Growth Chamber Conditions

General description: Inoculation of wheat with leaf rust, spraying of potentially bioactive compounds to control the infection, and procedure for evaluation of the infection level.

### Method:

### A. Preparation of Wheat Seedling for Inoculation

1) Use: seedling pots of size 90×80×80, standard garden earth with fertilizer and wheat seeds of a sensitive variety (from Beit Hashita farm, Israel).
2) Put 12 pots in a large tray and fill the pots with the earth to the top.
3) Make a grove for the seeds using a 250 ml bottle.
4) Put 10 seeds of wheat in each pot (in a circle).
5) Cover the seeds with additional earth and press strongly.
6) Add water into the tray—about 100 ml for each pot (fill the tray 3 times).
7) Grow the wheat for 2 weeks, in growth room at 24° C. prior to inoculation.

### B. Preparation of Spore's Suspension

1) Insert 30 wheat leaves with spores, into a sterile 50-ml tube.
2) Add 40 ml of cold 0.05% Tween® 20 solution.
3) Shake the tube on the vortex for 2 min at maximum speed.
4) Transfer the suspension (without the leaves), into a clean sterile 50-ml tube on ice.
5) Filter the spore suspension through 16-layer gauze cloth directly into a clean sterile 50-ml tube to discard the hyphae—about 30 ml should be recovered.
6) Wash the spores on a 5-micron pore membrane, to discard bacteria and other fungi spores —stop the vacuum pump and spray cold sterile water to suspend and wash the spores, start the vacuum pump again.
7) Repeat spore wash one more time.
8) Suspend the spores in a 50-ml tube with 10 ml sterile cold 0.05% Tween® 20 solution—insert the membrane with the spores into the tube with the Tween® 20 solution and shake the tube by hand.
9) Remove the membrane and discard it.
10) Decant filtered liquid to the sink and wash the filtration system with top water and dry it.
11) [Optional] Add 10 μl Chloramphenicol stock solution (20 mg/ml) for final concentration of 20 μg/ml.
12) Check the spore concentration in the suspension—the concentration should be about 30,000 spores/ml and should have a brown colour.
13) Dilute the spore suspension to get 4000 spores/ml using cold 0.05% Tween® 20 solution.
14) Keep the spore suspension on ice.

### C. Inoculation of Wheat Plants for Infection Experiments

1) Grow plants for about 2 weeks till the first leaf is fully developed.
2) Use pots with 9-10 plants.
3) Spray the spore suspension 4000 spores/ml on the wheat plants 0.1 ml per plant.
4) Use the compressor paint brush system with 0.5-mm orifice, at 40 PSI, to spray the spore suspension and spray 4 pots on a revolving tray at a time, do it twice.
5) Insert the pots with the wheat inoculated plants into a moist chamber at 20° C. [the hot water at 30° C. and the room temperature at 18° C.] for overnight.
6) Immediately after the moist chamber, put cylinders on the pots and transfer them to the growth room.
7) Grow the wheat in the growth room, at 24° C., with 16 h light/8 h dark regime.

### D. Analysis of Infection Level

1) Analysis of infection level is performed after about 2 weeks.
2) Infection level of the first leaf alone is analysed.
3) Infection level is scored according to leaf coverage of spore patches.
4) A 100% of spore patches coverage, should be decided before the experiment, a photo of such a leaf will be used for the assessment of the infection level.

### E. Application of the Tested Compound

1) Formulated Compound 1 treatment was given a day before inoculation via spraying.
2) Each plant was sprayed by 100 ul of formulated compound 1 (see Example 9).
3) Each treatment included 4 pots with 9-10 plants in each pot.

### Results

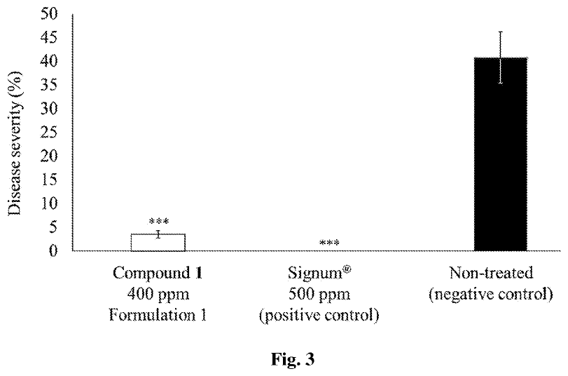

Two experiments were conducted under controlled environment in growth rooms where the bioactivity potential of Compound 1 to prevent and control Puccinia sorghi in corn plants was estimated (FIGS. 2, and 3). Compound 1 performed very well and showed very good efficacy under controlled growth conditions. The average efficacy of Compound 1 in preventing and controlling the Puccinia triticina was 95.17% at 200 ppm and 97.06% at 400 ppm.

### Example 12. Validation Experiments in Tomato Detached Leaves Infected with Phytophthora infestans

General description: Detached leaves of tomato were treated by 1-phenyl-tetralin compound and infected by spores of Phytophthora infestans.
Phytophthora spore suspension preparation: Prepare spores according to Example 6 and dilute by water to 1000 spores/ml.

### A. Preparation of Tomato Leaves for Inoculation:

1) Put two pieces of sterile paper in a square Petri dish.
2) Work in sterile conditions.
3) Use 3^{rd} to 5^{th} leaves from the top.
4) Add sterile distilled water to wet the paper.
5) Cut lobes from the leaves by a sterile scalpel.
6) Put 10 lobes of leaves in a square petri dish, on the wet paper, lower side of the leaf should face the paper.
7) Cover the plate with a lid.

### B. Treatment and Inoculation of Spores on Detached Leaves

1) Spray 1 ml of treatment on all the leaves in one square dish (using a spraying syringe) on the upper side of the leaf and let the leaves dry in the chemical hood.
2) Spray 1 ml of Phytophthora spores' suspension on all the leaves in one square dish (using a spraying tool) on the upper side of the leaf.
3) Cover the dish and seal it by stretched nylon, allow the fungal growth on the leaves according to Example 6 and record the level of infection after 7 days.
   Method for Formulations Used in Exps. 487, 492, 500 (with Reference to Tables 4-6 Below)

Compound 3 was dissolved in absolute ethanol with 1:36 weight to weight ratio or in dimethyl-sulfoxide with 1:17 weight to weight ratio, sonicated for 5 mins and then another part of non-ionic detergent either Tween® 20 with weight to weight 1:4.5 ratio to Compound 3 or Silwet® in 1:1 weight to weight ratio to Compound 3 was added for formulation finalisation. In some cases, sodium carbonate (Na₂CO₃) was used to adjust pH to 6.

### Results

Three independent experiments were conducted in detached tomato leaves where the bioactivity potential of Compound 3 to prevent and control Phytophthora infestans was estimated (see the results in Tables 4-6 below). The severity of infection by Phytophthora was evaluated using the following grades which expresses the leaf area covered by fungus: 0=clear; 1=low coverage; 2=medium coverage; 3=high coverage.

Compound 3 controlled the Phytophthora infection with the efficacies between 73% to 83% at 200 ppm.

TABLE 4
Exp. 487: Effect of Compound 3 on tomato leaf infection
determined as leaf surface area (0-3) covered by Phytophthora

| | | T-test vs. |
|---|---|---|
| | | Phytophthora |
| Treatment | Mean | inoculated, |
| (all including Phytophthora) | (n = 10) | not treated |
| Compound 3 (200 ppm in DMSO | 0.1 | <0.001 |
| 6% + Silwet ® 0.005% | | |
| Compound 3 (200 ppm in 0.1% base | 0.7 | <0.001 |
| (pH = 6) + Silwet ® 0.005% | | |
| Silwet ® 0.005% | 2.9 | n.s. |
| Phytophthora inoculated, not treated | 2.6 | |

TABLE 5
Exp. 492: Effect of Compound 3 on tomato leaf infection
determined as leaf surface area (0-3) covered by Phytophthora

| | | T-test vs. |
|---|---|---|
| | | Phytophthora |
| Treatment | Mean | inoculated, |
| (all including Phytophthora) | (n = 10) | not treated |
| Compound 3 (200 ppm in DMSO | 0.4 | <0.001 |
| 6% + Silwet ® 0.005% | | |
| Compound 3 (200 ppm in 0.1% | 0.3 | <0.001 |
| base (pH = 6) + Silwet ® 0.005% | | |
| Silwet ® 0.005% | 2.3 | n.s. |
| Phytophthora inoculated, not treated | 3 | |

TABLE 6
Exp. 500: Effect of Compound 3 on tomato leaf infection
determined as leaf surface area (0-3) covered by Phytophthora

| Treatment | Mean | T-test vs. Phytophthora |
|---|---|---|
| (all including Phytophthora) | (n = 10) | inoculated, not treated |
| Compound 3 (200 ppm in 0.1% | 0.8 | <0.001 |
| base (pH = 6) without Silwet ® | | |
| Silwet ® 0.005% | 3 | n.s. |
| Phytophthora inoculated, not | 3 | |
| treated | | |

### Example 13. Validation In-Vivo Experiments in Wheat Infected with Puccinia trititcina Under Greenhouse Conditions

General description: Leaves of wheat, with colonies of leaf rust were sprayed by formulated Compound 3 (treatment) and the percentage of spore germination was evaluated in three time points following the treatment (1, 7 and 14 d).
Sub protocol: 1) Pustules germination; 2) Inoculation of wheat with Puccinia.

### Method:

1) Wheat plants, from a sensitive cultivar (Beit Hashita, Hazera, Israel) were pre-inoculated with Puccinia triticina and transferred to the greenhouse or grown/greenhouse/field. Colonies/pustules from different age and maturity were developed.
2) Formulated Compound 3 was applied in the relevant concentrations, until full drainage of the leaves (5 ml/pot). See below for details.
3) At id, 7 d, 14 d following treatment, collect leaves and store at the humid chamber and send to analysis to the lab.
4) Preparation of 96 well plate for spore germination assay:
   a) Cut from the collected leaves 20 colonies (pustules)/treatment. Each colony should be cut separately with a lab scalpel.
   b) Use a 96 well plate for the spore germination.
   c) Insert one pustule of Puccinia into each well. Make sure number of colonies tested from each treatment is 20.
   d) As a control—take leaves of lab grown P. triticina developed on wheat and not-treated with Compound 3.
   e) Fill each well with 150 μl of 0.05% Tween® 20 solution.
   f) Seal the plate with a plate cover and put it on shaker for 10 min at 2000 rpm.
   g) Take out the leaves from each well.
   h) Take out 120 μl of the solution, and leave 25-30 ul in each well.
   i) Do not shake the plate!
5) Incubate at 17° C. dark for overnight.
6) At the next day—check spore germinated in the control samples.
7) Count the number of germinating spores/out of 10 spores in each well, using the microscope (×10 magnitude). Do not count all spores in each well, rather select only 10 spores to measure, and count germinated spores out of the 10 selected.
8) Calculate the average percentage of germinating spores/treatment.
9) Perform average of all 20 samples/treatment.
10) Perform statistical analysis comparing to non-treated control.
   A. Preparation of Wheat Seedling for Generation of P. triticina Pre-Inoculated Plants in Pots
1) Use seedling pots of size 120×80×80.
2) Use standard garden soil mix with fertilizer (coconut 50%, pit 44%, and quartz 6% with starter 18-24-5 fertilizer 5 kg/m³ and slow release 14-14-14 fertilizer).
3) Use wheat seeds of a sensitive variety (used from Beit Hashita farm).
4) Put 12 pots in a large tray and fill the pots with the soil to the top and press it a little.
5) Make a grove for the seeds.
6) Put 12 seeds of corn in each pot (in a circle), cover the seeds with additional soil mix and press strongly.
7) Add water into the tray—about 100 ml for each pot (fill the tray 3 times).
8) Grow the wheat for 3 weeks, in growth room at 24° C. before inoculation.
9) Move the wheat seedlings for further growth in the greenhouse.
10) Grow the seedlings in the clean area (no disease or inoculated plants around).
   B. Preparation of Pre-Inoculated P. triticina Wheat Plants
1) Insert 30-40 infected wheat leaves with spores, into a sterile 50-ml tube.
2) Add 40 ml of cold 0.05% Tween® 20 solution.
3) Shake the tube on the vortex for 2 min at maximum speed.
4) Transfer the suspension (without the leaves) into a clean sterile 50-ml tube on ice.
5) Filter the spore suspension through 16-layers gauze cloth directly into a clean sterile 50-ml tube to discard the hyphae (about 30 ml should be recovered).
6) Wash the spores using a vacuum pump on a 5-micron pore membrane, to discard bacteria and other fungi spores—stop the vacuum pump, and spray cold sterile water to suspend and wash the spores, then start the vacuum pump again.
7) Repeat spore wash one more time.
8) Take the membrane, carefully, from the pump into new 50-ml tube and suspend the spores with 10 ml sterile cold 0.05% Tween® 20 solution and shake the tube by hand to release the spores from the membrane.
9) Remove the membrane and discard it.
10) Decant filtered liquid to the sink and wash the filtration system with top water and dry it.
11) [Optional] Add 10 μl Chloramphenicol stock solution (20 mg/ml) for final concentration of 20 μg/ml.
12) Check the spore concentration in the suspension using haemocytometer under a microscope. The concentration should be about 30,000 spores/ml and the spores should have a brown colour.
13) Dilute the spore suspension to get 4000 spores/ml using cold 0.05% Tween® 20 solution.
14) Keep the spore suspension on ice.
15) Spray the 3 weeks wheat seedlings with the spore suspension (1 ml/seedling).
16) Move the sprayed seedlings into dark humid chamber for overnight.
17) Take plants out of dark humid box. Cover each pot with a clear plastic cylinder, to keep moist around the wheat leaves. Move the infected seedlings into growth chamber, for further development.
18) Pustules of P. triticina should be observed on the wheat leaves within 7-10 days.

### Formulation Preparation

See formulation 2 preparation in Formulation section in Example 9.

### Results

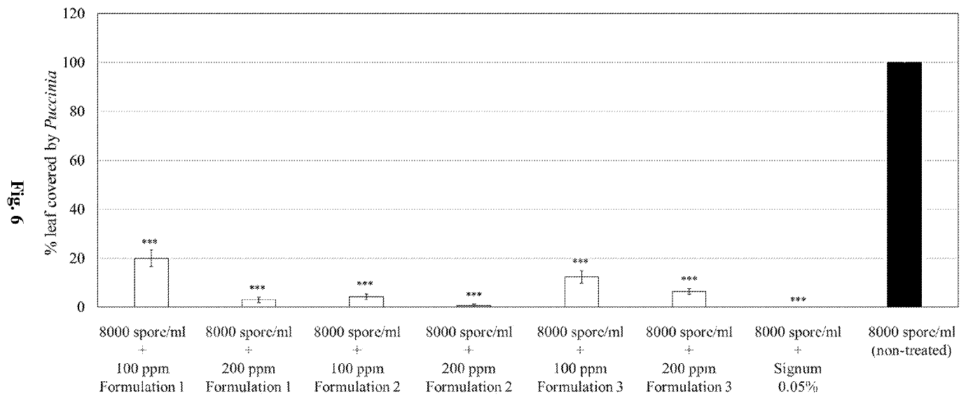

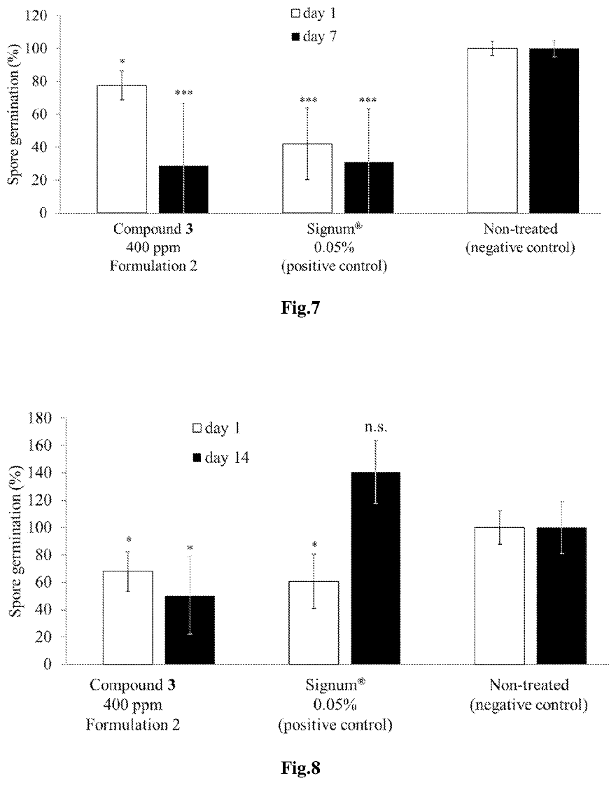

Three experiments were conducted under greenhouse conditions where the bioactivity potential of Compound 3 to inhibit Puccinia triticina spore's germination was estimated (FIGS. 7-10). Compound 3 performed very well and showed very good efficacy under greenhouse conditions. The efficacy of Compound 3 in inhibiting Puccinia triticina spore germination was up to 72.8% at 400 ppm.

### Example 14. Validation In-Vivo Experiments in Tomato Infected with Phytophthora infestans Under Greenhouse Conditions

General description: Severity of late blight disease caused by Phytophtora infestans was evaluated following treatment with 1-phenyl-tetralin derivatives. Sporangium was used to infect 3-4 weeks-old tomato young plants following curative treatment with the 1-phenyl-tetralin derivatives.

### A. Pathogen Sporangium Preparation (Grown on Plates/Solid Media)

### Preparation of Sporangium Suspension

1) Put 10 lobes of 4 days old Phytophthora infected tomato leaves, in a sterile 50 ml tube.
2) Fill the tube with 40 ml of 4 ml of the cold sterile distilled water
3) Mix the tube gently, by hand, to release the sporangium into the water, but avoid damaging the leaf tissue
4) Filter the spore suspension through 16 layers of miracloth into 50-ml tube.
5) Calculate the spore concentration using a microscope with 200× magnification, which is expected to be 3000 sporangium/ml.
6) Chill the tube on ice.

### B. Sporangium Washing and Concentration by Filtration

1) Prepare filtration system with filter membrane (in range of 0.45 μM to 5 μM pore size) and wash the membrane with sterile cold water.
2) Suspend and decant the spore suspension from the 50-ml tube slowly into the filtration system. Use low vacuum, do not let the membrane get dry—leave 4 ml unfiltered suspension on the filter.
3) Wash the spore to discard bacteria and other fungal spores with 40 ml of sterile cooled distilled water.
4) Repeat 5 times the washing process. Make sure the membrane will not get dry between the washing steps.
5) Collect the spore suspension into a clean 50-ml tube.
6) Insert the membrane using for filtration into the tube with the sporangium and gently suspend the sporangium left on the membrane.
7) Discard the membrane and wash and sterilize the filtration-vacuum system by hypochlorite solution (0.1%)—allow to stand for at least 1 hour in the hypochlorite solution.
8) Calculate sporangium concentration—using a microscope haemocytometer slide with ×200 magnification. The final concentration needed for inoculation is 6000 spore/ml.
9) Store the sporangium in the fridge.

### C. Plant/Seedling Germination Conditions

1) Tomato Ikram/Brigade/Shani cultivars (sensitive to Phytophthora) were germinated in seedlings tray using standard greenhouse soil mixture. Seedlings were grown in clean growing chamber with 24° C. temperature under 12 h light/12 h dark regime. Seedlings of 3-4 weeks old with 4 true leaves were used for experiments
2) Plants needed for each treatment were transferred from the seedling’ trays to a dedicated experimental tray.
3) Two leaves on each seedling were labelled by small plastic tags. On each labelled leaf the last largest 3 leaflets were used for the experiment.

### D. Inoculum Application (Curative Approach)

1) Young tomato seedlings were moved to the greenhouse for the experimental procedure.
2) In the curative approach, inoculum was applied, then 24 hours following inoculation, the treatment was applied:
   a) 10 μl drop of Phytophtora infestans was applied on each labelled leaflet.
   b) The tray with the labelled inoculated leaves was put into humid dark box, with low level of water at the bottom of the darkened box. The box was kept at 17° C. for 24 h.
   c) After 24 h, the tray with the inoculated plants was moved to greenhouse table to allow disease to develop.

### E. Treatment Application (Curative Approach)

1) 1-phenyl-tetralin compound was applied 24 h following inoculation. Plants were removed from the humid box.
2) Formulated 1-phenyl-tetralin compound was applied using hand sprayer, until full drainage of the tomato leaves. The treatment was applied on the upper and lower side of the leaves.
3) 3.5 ml of formulated treatment was applied per each 2 plants.
4) After 24 h following the first treatment, the treatment was applied again.

### F. Inoculation Application (Preventative Approach)

In the preventative approach, inoculation was applied following two repeating treatments with Compound 3:
a) 10 μl drop of Phytophthora infestans freshly prepared (6000 spore/ml) spores' suspension was applied on each labelled leaflet (6 leaflets on each plant).
b) Inoculated plants were put into a humid box for 24 h.
c) After 24 h, the tray with the inoculated plants were removed from the humid box and placed in the greenhouse for further growth and disease development.

### G. Treatment Application (Preventative Approach)

1) 4 weeks-old healthy tomato seedlings with 4 true leaves were used. The last three leaflets of two mature leaves on each plant were labelled with a small plastic tag.
2) 48 h before inoculation (Day −2), plants were treated with formulated Compound 3 and respective control treatments using hand sprayer until the full drainage of the tomato leaves (1 ml/plant) on the upper and bottom side of the leaf.
3) 24 h before inoculation (Day −1), the plants were treated again with the same treatments.

### H. Growth and Analysis

1) Following treatment and inoculations, plants were grown under normal greenhouse conditions and watered according to need.
2) Five days following inoculation, disease was observed on the labelled leaves.
3) The labelled leaves were cut and collected, each treatment separately, and moved to the lab to measure the decay percentage expressed as disease severity in %.
4) Late blight symptoms should be observed as brownish-green spots which appear on the infected spot, then large areas of the leaves turn brown completely.

### Formulation Preparation

See formulation preparation in Formulation section in Example 9.

### Results

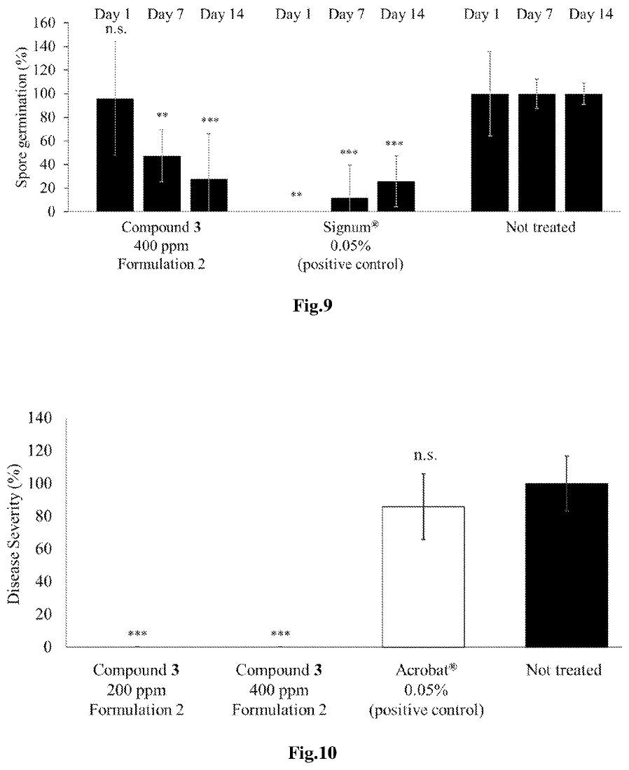

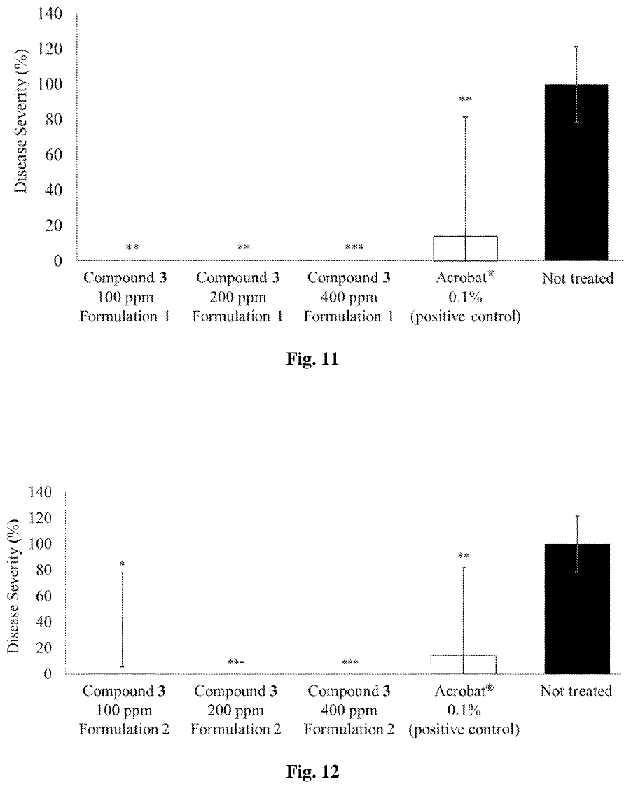

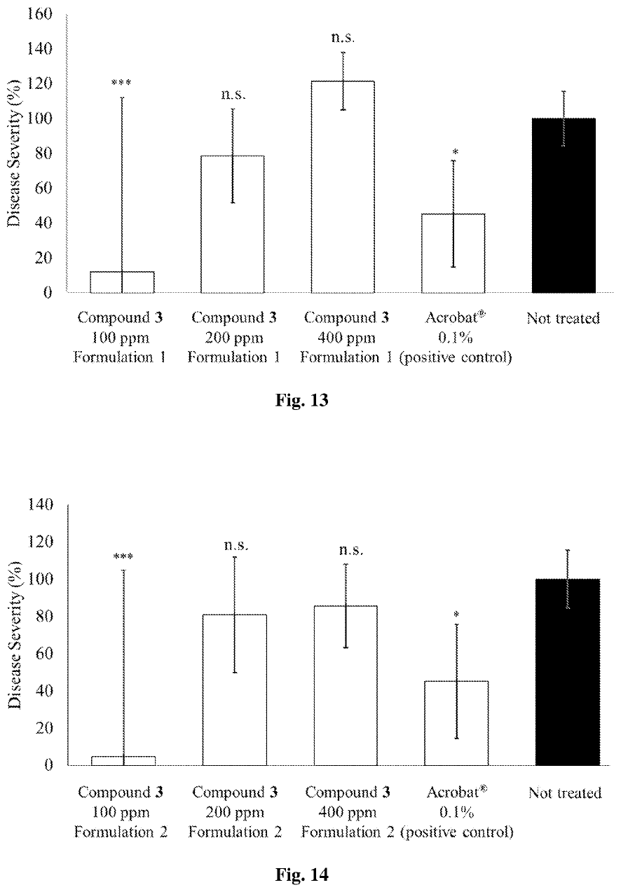

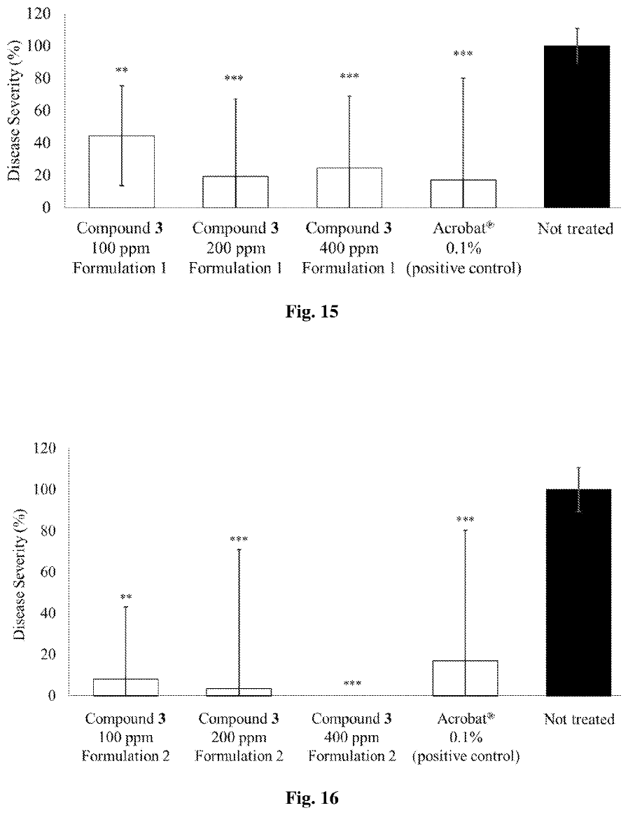

Seven independent experiments were conducted in tomato plants infected with Phytophthora, where the potential of Compound 3 to prevent and control Phytophthora infestans (FIGS. 10-16) was estimated.

Compound 3 controlled the Phytophthora infection with the efficacy up to 100%.

### Example 15. Validation In-Vivo Experiments in Tomato Infected with Alternaria solani Under Greenhouse Conditions

General description: Severity of early blight disease caused by Alternaria solani was evaluated following treatment with Compound 3. Spores isolates were used to infect leaves of 3-4 weeks old tomato young plants following preventative treatment by 1-phenyl-tertralin compounds.

### A. Alternaria Spore Suspension Preparation

1) Put a PDAT block of Alternaria in the middle of a PDAT plate and grow for nine or more days at 25° C.
2) Add 25 ml of fridge cold, sterile, PDB to a 50-ml tube.
3) Cut the agar with the hyphae and spores from one plate to 8 pieces by scalpel and insert them into the 50-ml sterile tube.
4) Shake for 1 min.
5) Keep spores on ice during the whole process.
6) Transfer the liquid to a new 50-ml sterile tube—about 25 ml should be recovered.
7) Filter the spore suspension through 16-layers gauze cloth directly into a clean sterile 50-ml tube to discard the hyphae—about 20 ml should be recovered.
8) Calculate the spore concentration (count ×10 dilution at 20×10 magnification) and record it.
9) The concentration should be 10⁴-10⁵ spore/ml.

### B. Tomato Plants Preparation

1) Tomato Ikram/Brigade/Shani cultivars, susceptible to the Alternaria were germinated four weeks prior to the experiment in a seedlings tray using general greenhouse soil mixture (composition of soil is: 44% pith, 50% coconut, 6% quartz, with long range fertilizer [osmocote 14:14:14] and NPK fertilizer 18:24:5 5 kg/M³). Germination and growth of the seedlings is performed in clean growing chamber 24° C. temperature, with 12 h light/12 h dark regime. Seedlings of 3-4 weeks old, with 4 true leaves are used for experiments.
2) Plants needed for the experiment were transferred to a dedicated experimental tray.
3) Two leaves on each tomato plant were labelled by small plastic tags. On each labelled leaf last largest 3 leaflets were used for the experiment.

### C. Treatment Application (Preventative Approach)

1) 4-weeks old tomato seedlings with 4 true leaves, clean and healthy were moved to the greenhouse for the experimental procedure.
2) 48 h before inoculation (Day −2), plants were treated with the appropriate treatments according to the experimental plan.
3) The experimental plan included Compound 3, which was applied in the above indicated concentration. A chemical reference treatment and non-treated plants also were included in the experiment.
4) Formulated Compound 3 was applied to plants via spraying, using hand sprayer, until full drainage of the tomato leaves (1 ml/plant). Treatment was applied on the upper and lower sides of the leaves.
5) 24 h before inoculation (Day −1), a second treatment was applied to the plants.
6) Calculation of averages, standard-error and statistical analysis was performed.

### D. Growth and Analysis

1) Following inoculation and treatment, plants were moved to grow in normal greenhouse condition, with watering regime, as needed, according to the season.
2) 10-14 days after inoculation, disease was observed on the labelled leaves.
3) The labelled leaves are collected from each treatment separately, and moved to the lab to collect data on lesion development.

4) Early blight symptoms (Alternaria) were observed as yellow-brownish spots that appeared on the infected spot. The yellow-brown diameter of the decay on each labelled leaflet was measured. The total leaflet size was measured as well.
5) The yellow-brown diameter of the decay on each labelled leaflet and disease severity were estimated as percentage of decay area out of total labelled leaflet area.

### Formulation Preparation

See Formulation 2 preparation in Formulation section in Example 9.

### Results

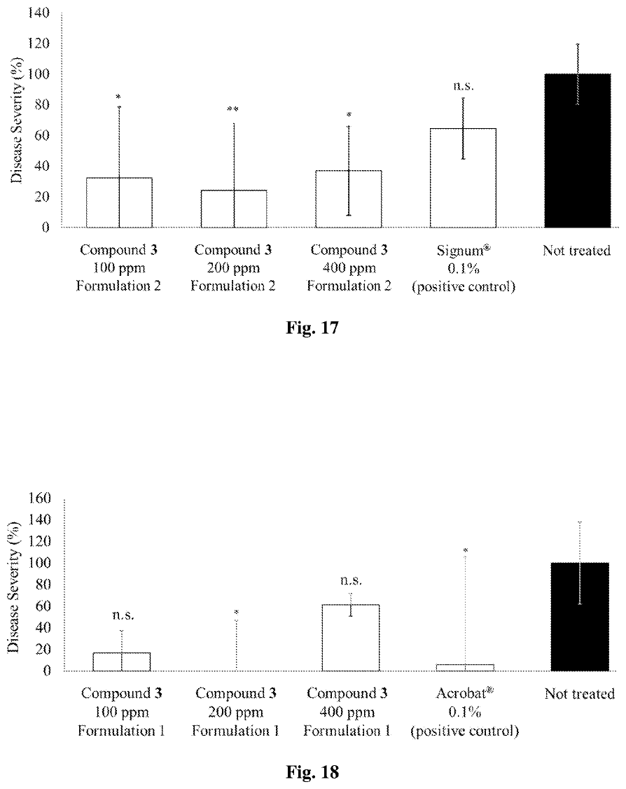

Experiments were conducted in tomato plants infected with Alternaria, where the bioactivity potential of Compound 3 to prevent and control Alternaria solani was estimated (see FIG. 17). Compound 3 controlled the Alternaria infection with the efficacy up to 75.8%.

### Example 16. Validation In-Vivo Experiments in Tomato Infected with Botrytis cinerea Under Greenhouse Conditions

### A. Botrytis Spore Suspension Preparation

1) Put a PDAT block of Botrytis in the middle of a small petri PDAT plate and grow for 12 days at 23° C. Keep the plate with the cover upside up (so the drought will affect and encourage sporulation). The B. cinerea hyphae should propagate (white-light grey colour) and develop the spores on the inoculum (grey colour), after the conidia was growing throughout all the plate.
2) Chill the plate in the fridge for 1 h.
3) Cut the agar with the hyphae and spores from one plate to 8 pieces by scalpel and insert them into a 50-ml sterile tube.
4) Add 25 ml of fridge cold, sterile, 8×-PDB solution to the tube.
5) Shake for 1 min at 3000 RPM.
6) Keep spores on ice during the process.
7) Transfer the liquid to a new 50-ml sterile tube—about 25 ml should be recovered.
8) Filter the spore suspension through 16-layers gauze cloth directly into a clean sterile 50 ml tube to discard the hyphae—about 20 ml should be recovered.
9) Calculate the spore concentration (count ×10 dilution at 40×10 magnification) and dilute by cold sterile 8×-PDB solution to get 2×10⁵ spores/ml stock (the concentration before dilution is expected to be 3×10⁵).
10) Dilute the spore suspension stock with 8×-PDB solution to get inoculation spore suspension −1×10⁵ spores/ml.
11) Use immediately to infect tomato leaves or store at 4° C. for maximum 1 week.

### B. Plant/Seedling Germination Conditions

1) Tomato Ikram/Brigade/Shani cultivars were germinated in seedlings tray using general greenhouse soil mixture (composition of soil is: 44% Pith, 50% coconut, 6% quartz, with long range fertilizer [osmocote 14:14:14] and NPK fertilizer 18:24:5 5 kg/M³). Germination and growth of the seedlings is performed in clean growing chamber with 24° C. temperature and with 12 h light/12 h dark regime. 3-4 weeks old seedlings with 4 true leaves were used for experiments.
2) Plants needed for each treatment were transferred to a dedicated experimental tray.
3) Two leaves on each seedling were labelled by small plastic tags. Only the last 3 leaflets were used for the experiment.

### C. Treatment Application (Preventative Approach)

4) 4-weeks old tomato seedlings with 4 true leaves, clean and healthy were moved to the greenhouse for the experimental procedure. The last three leaflets of two mature leaves on each plant were labelled with plastic tags.
5) 48 h before inoculation (Day −2), plants were treated with formulated Compound 3 with the appropriate concentration according to the experimental plan.
6) A chemical reference treatment and non-treated plants also were included in the experiment.
7) Formulated Compound 3 was applied to plants using hand sprayer until full drainage of the tomato leaves (5 ml/2 plants). The treatments were applied on the upper and lower sides of the leaves.
8) 24 h before inoculation (Day −1), a second spraying was applied to the plants.

### D. Inoculation Application

1) Mark a black point with a thin marker on each labelled leaflet
2) Use a 200-μ1 tip and gently make a small wound, without tearing the leaf, close to the labelled point.
3) In the curative approach, inoculation is applied, then after 72 h following inoculation, the following MI treatment is applied:
a) On each leaflet, apply a 10 μl drop of Botrytis cinerea on the black point, in each labelled leaflet. Allow to stay for 30 min for the drop to be soaked in the tissue.
b) Insert the tray with the labelled inoculated plants to a humid box, with low level of water at the bottom of the box. Keep the inoculated plants in the box for 72 h.
c) After 3 days, open gently the box lid (half open) to allow slow balance of the different humidity level between the box and the environment. Following humidity balance, take the tray of plants out, and apply the treatment.

### E. Treatment Application (Curative Approach)

1) Treatments were applied 72 h following inoculation.
2) Formulated Compound 3 was applied to plants using a hand sprayer until full drainage of the tomato leaves. The treatments were applied to the upper and lower sides of the leaves.

### F. Growth and Analysis

1) Following treatment and inoculations, plants were moved to the growing table and maintained under normal greenhouse conditions with watering regime as needed according to the season.
2) 10-14 days following inoculation, disease was observed on the labelled leaves. 3) The labelled leaves were collected from each treatment separately and transferred to the lab to measure each leaflet and decay size.
4) Grey mold (Botrytis) symptoms were observed on the old leaves and had green or green-yellow stains, that will develop to necrosis.
5) The lesion diameter of each leaflet and the leaflet size was measured.
6) Calculation of averages, standard-error and statistical analysis was performed.

### Formulation Preparation

See Formulations 1 and 2 preparation in Formulations section in Example 9.

### Results

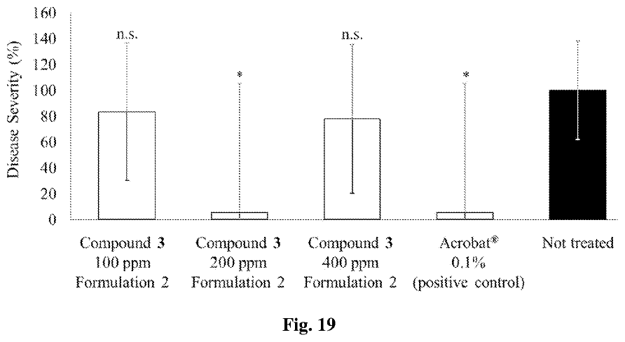

Two independent experiments were conducted in tomato plants infected with Botrytis where the potential of Compound 3 to prevent and control Botrytis cinerea was estimated (see FIGS. 18-19). Compound 3 controlled the Botrytis infection with the efficacy up to 100%.

### REFERENCES

Erlacher A., Cardinale M., Grosch R., Grube M., Berg G. The impact of the pathogen Rhizoctonia solani and its beneficial counterpart Bacillus amyloliquefaciens on the indigenous lettuce microbiome. Front Microbiol. 2014; 5: 175. Published online 2014 Apr. 21. doi: 10.3389/fmicb.2014.00175.
Mesfin Kebede Gessese. Description of Wheat Rusts and Their Virulence Variations Determined through Annual Pathotype Surveys and Controlled Multi-Pathotype Tests. Advances in Agriculture, 2019; Article ID 2673706.
Groth, J. V., Zeyen, R. J., Davis, D. W., & Christ, B. J. (1983). Yield and quality losses caused by common rust (Puccinia sorghi Schw.) in sweet corn (Zea mays) hybrids. Crop Protection, 2(1), 105-111.
Hershman D. E., Sikora E. J., Giesler L. J. Soybean Rust PIPE: Past, Present, and Future. Journal of Integrated Pest Management, Volume 2, Issue 2, 1 Oct. 2011, pp D1—D7, https://doi.org/10.1603/IPM11001.
Hofte M. and De Vos P. Plant pathogenic pseudomonas species. Gnanamanickam S. S. (ed.), Plant-Associated Bacteria, 2006; 507-533.
J. A. L. van Kan, Infection Strategies of Botrytis cinerea. Proc. VIIIth IS Postharvest Phys. Ornamentals. Acta Hort. 669, ISHS 2005.
Jenkins J. E. E Clark Y. S. and Buckle A. E. Fusarium diseases of cereals. Research Review 4. October 1988.
Frank N. Martin & Joyce E. Loper. Soilborne Plant Diseases Caused by Pythium spp.: Ecology, Epidemiology, and Prospects for Biological Control. Critical Reviews in Plant Sciences, 1999; 18:111-181.
Moore. L. W. Pseudomonas syringae: disease and ice nucleation activity. Ornamentals Northwest Newsletter. (1988) 12:4-16.
Patriarca, A., & Fernández Pinto, V. (2018). Alternaria⋆. Reference Module in Food Science. doi:10.1016/b978-0-08-100596-5.22572-9
Sedláková V., Dejmalová J., Hausvater E., Sedlák P., Doležal P. & Mazáková J. Effect of Phytophthora infestans on potato yield in dependence on variety characteristics and fungicide control. Plant Soil Environ., 57, 2011 (10): 486-491.

1. A method for controlling, preventing, reducing or eradicating instances of plant-pathogen infestation on a plant, plant organ, plant part, or plant propagation material, the method comprising: applying to a plant, plant part, plant organ or plant propagation material, or to soil surrounding said plant, a pesticidally effective amount of at least one compound of formula (I): 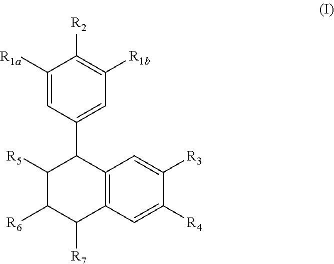
wherein R₁ₐ, R_{1b}, R₂, R₃ and R₄ are independently selected from hydrogen, methyl, hydroxy and methoxy group, and halogen atom (F, Cl, Br, I);
R₅ and R₆ are independently selected from hydrogen, methyl and ethyl; and
R₇ is selected from hydrogen, methyl, amino, methylamino, dimethylamino, hydroxy and methoxy,
or stereoisomers, or agriculturally acceptable salts thereof.

2. The method of claim 1, wherein R₁ₐ, R_{1b}, R₂ are independently selected from hydrogen and halogen atom (F, Cl, Br, I);
R₃, R₄, R₅ and R₆ are hydrogen; and
R₇ is selected from hydrogen, methyl, amino, methylamino, dimethylamino, hydroxy and methoxy group.

3. The method of claim 2, wherein R₁ₐ, R_{1b}, R₂ are independently selected from hydrogen and chlorine atom;
R₃, R₄, R₅ and R₆ are hydrogen; and
R₇ is methylamino group.

4. The method of claim 3, wherein said compound is (1S,4R)-4-(3,4-dichlorophenyl)-N-methyl-1,2,3,4-tetrahydronaphthalen-1-aminium chloride.

5. The method of claim 1, wherein R₁ₐ, R_{1b}, R₂, R₃ and R₄ are independently selected from hydrogen, methyl, hydroxy and methoxy group, and halogen atom (F, Cl, Br, I);
R₅ and R₆ are methyl; and
R₇ is selected from hydrogen, methyl, amino, methylamino, dimethylamino, hydroxy and methoxy group.

6. The method of claim 5, wherein R₁ₐ, R_{1b}, R₂, R₃ and R₄ are independently selected from hydrogen, hydroxy, and methoxy group;
R₅ and R₆ are methyl; and
R₇ is selected from hydrogen, methyl, amino, methylamino, dimethylamino, hydroxy and methoxy group.

7. The method of claim 6, wherein R₁ₐ, R_{1b}, R₂, R₃ and R₄ are independently selected from hydrogen, hydroxy, and methoxy;
R₅ and R₆ are methyl; and
R₇ is hydrogen.

8. The method of claim 7, wherein said compound is selected from:
5-(3,4-dihydroxyphenyl)-6,7-dimethyl-5,6,7,8-tetrahydronaphthalene-2,3-diol;
(5R,6R,7R)-5-(3,4-dihydroxyphenyl)-6,7-dimethyl-5,6,7,8-tetrahydronaphthalene-2,3-diol;
4-(7-hydroxy-6-methoxy-2,3-dimethyl-1,2,3,4-tetrahydronaphthalen-1-yl)benzene-1,2-diol; and
4-((1R,2R,3R)-7-hydroxy-6-methoxy-2,3-dimethyl-1,2,3,4-tetrahydronaphthalen-1-yl)benzene-1,2-diol,
or combinations thereof.

9. The method of claim 8, wherein said compound is (5R,6R,7R)-5-(3,4-dihydroxyphenyl)-6,7-dimethyl-5,6,7,8-tetrahydronaphthalene-2,3-diol.

10. The method of claim 8, wherein said compound is 4-((1R,2R,3R)-7-hydroxy-6-methoxy-2,3-dimethyl-1,2,3,4-tetrahydronaphthalen-1-yl)benzene-1,2-diol.

11. The method of claim 4, wherein the plant-pathogen to which said compound is applied is a member selected from: a Basidomycete of the class Pucciniomycetes or the genus Rhizoctonia; an Ascomycota of the class Dothideomycetes or a genus selected from Botrytis and Fusarium; and a Heterokontophyta of the class Oomycota.

12. The method of claim 11, wherein said plant-pathogen is a member of the class Pucciniomycetes plant-pathogen of the order Pucciniales.

13. The method of claim 12, wherein said Pucciniales plant-pathogen is a member of the family Pucciniaceae.

14. The method of claim 13, wherein said Pucciniaceae plant-pathogen is a member of the genus Puccinia spp.

15. The method of claim 14, wherein said plant-pathogen is selected from Puccinia sorghi and Puccinia triticina.

16. The method of claim 11, wherein said plant-pathogen is a member of the genus Rhizoctonia of the species Rhizoctonia solani.

17. The method of claim 11, wherein said plant-pathogen is a member of the class Dothideomycetes of the order Pleosporales.

18. The method of claim 17, wherein said Pleosporales plant-pathogen is a member of the family Pleosporaceae.

19. The method of claim 18, wherein said Pleosporaceae plant-pathogen is a member of the genus Alternaria.

20. The method of claim 19, wherein said Alternaria plant-pathogen is selected from Alternaria alternata and Alternaria solani.

21. The method of claim 11, wherein said plant-pathogen is a member of the genus Botrytis of the species Botrytis cinerea.

22. The method of claim 11, wherein said plant-pathogen is a member of the genus Fusarium of the species Fusarium oxysporum.

23. The method of claim 11, wherein said plant-pathogen is a member of the class Oomycota of the order Peronosporales.

24. The method of claim 23, wherein said Peronosporales plant-pathogen is a member of the family Peronosporaceae or Pythiaceae.

25. The method of claim 24, wherein said Peronosporales plant-pathogen is a member of the family Peronosporaceae of the genus Phytophthora.

26. The method of claim 25, wherein said Phytophthora plant-pathogen is Phytophthora infestans.

27. The method of claim 24, wherein said Peronosporales plant-pathogen is a member of the family Pythiaceae of the genus Pythium.

28. The method of claim 27, wherein said Pythium plant-pathogen is Pythium aphanidermatum.

29. The method of claim 9, wherein the plant-pathogen to which said compound is applied is a member selected from: a Basidomycete of the class Pucciniomycetes or the genus Rhizoctonia; a Heterokontophyta of the class Oomycota; and a protobacterium of the order Pseudomonadales.

30. The method of claim 29, wherein said plant-pathogen is a member of the class Pucciniomycetes plant-pathogen of the order Pucciniales.

31. The method of claim 30, wherein said Pucciniales plant-pathogen is a member of the family Pucciniaceae.

32. The method of claim 31, wherein said Pucciniaceae plant-pathogen is a member of the genus Puccinia spp.

33. The method of claim 32, wherein said plant-pathogen is selected from Puccinia sorghi and Puccinia triticina.

34. The method of claim 29, wherein said plant-pathogen is a member of the genus Rhizoctonia of the species Rhizoctonia solani.

35. The method of claim 29, wherein said plant-pathogen is a member of the class Oomycota of the order Peronosporales.

36. The method of claim 35, wherein said Peronosporales plant-pathogen is a member of the family Peronosporaceae or Pythiaceae.

37. The method of claim 36, wherein said Peronosporales plant-pathogen is a member of the family Peronosporaceae of the genus Phytophthora.

38. The method of claim 37, wherein said Phytophthora plant-pathogen is Phytophthora infestans.

39. The method of claim 36, wherein said Peronosporales plant-pathogen is a member of the family Pythiaceae of the genus Pythium.

40. The method of claim 39, wherein said Pythium plant-pathogen is Pythium aphanidermatum.

41. The method of claim 29, wherein said plant-pathogen is a member of the order Pseudomonadales of the family Pseudomonadaceae.

42. The method of claim 41, wherein said Pseudomonadaceae plant-pathogen is of the genus Pseudomonas.

43. The method of claim 42, wherein said plant-pathogen is Pseudomonas syringae.

44. The method of claim 10, wherein the plant-pathogen to which said compound is applied is a member selected from: a Basidomycete of the class Pucciniomycetes or the genus Rhizoctonia; a Heterokontophyta of the family Pythiaceae; and a protobacterium of the order Pseudomonadales.

45. The method of claim 44, wherein said plant-pathogen is a member of the class Pucciniomycetes plant-pathogen of the order Pucciniales.

46. The method of claim 45, wherein said Pucciniales plant-pathogen is a member of the family Pucciniaceae.

47. The method of claim 46, wherein said Pucciniaceae plant-pathogen is a member of the genus Puccinia spp.

48. The method of claim 47, wherein said plant-pathogen is selected from Puccinia sorghi and Puccinia triticina.

49. The method of claim 33, wherein said plant-pathogen is a member of the genus Rhizoctonia of the species Rhizoctonia solani.

50. The method of claim 44, wherein said plant-pathogen is a member of the family Pythiaceae of the of the genus Pythium.

51. The method of claim 50, wherein said Pythium plant-pathogen is Pythium aphanidermatum.

52. The method of claim 44, wherein said plant-pathogen is a member of the order Pseudomonadales of the family Pseudomonadaceae.

53. The method of claim 52, wherein said Pseudomonadaceae plant-pathogen is of the genus Pseudomonas.

54. The method of claim 53, wherein said plant-pathogen is Pseudomonas syringae.

55. A pesticide composition comprising at least one compound of formula (I), 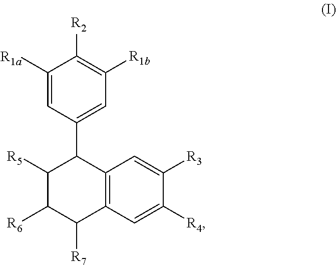
wherein R₁ₐ, R_{1b}, R₂, R₃ and R₄ are independently selected from hydrogen, methyl, hydroxy and methoxy group, and halogen atom (F, Cl, Br, I);
R₅ and R₆ are independently selected from hydrogen, methyl and ethyl; and
R₇ is selected from hydrogen, methyl, amino, methylamino, dimethylamino, hydroxy and methoxy group;
stereoisomers or agriculturally acceptable salts thereof.

56. The pesticide composition of claim 55,
wherein R₁ₐ, R_{1b}, R₂ are independently selected from hydrogen and halogen atom (F, Cl, Br, I);
R₃, R₄, R₅ and R₆ are hydrogen; and
R₇ is selected from hydrogen, methyl, amino, methylamino, dimethylamino, hydroxy and methoxy group.

57. The pesticide composition of claim 56,
wherein R₁ₐ, R_{1b}, R₂ are independently selected from hydrogen and chlorine atom;
R₃, R₄, R₅ and R₆ are hydrogen; and
R₇ is methylamino group.

58. The pesticide composition of claim 57, wherein said compound of formula (I) is (1S,4R)-4-(3,4-dichlorophenyl)-N-methyl-1,2,3,4-tetrahydronaphthalen-1-aminium chloride.

59. The pesticide composition of claim 55,
wherein R₁ₐ, R_{1b}, R₂, R₃ and R₄ are independently selected from hydrogen, methyl, hydroxy and methoxy group, and halogen atom (F, Cl, Br, I);
R₅ and R₆ are methyl; and
R₇ is selected from hydrogen, methyl, amino, methylamino, dimethylamino, hydroxy and methoxy group.

60. The pesticide composition of claim 59,
wherein R₁ₐ, R_{1b}, R₂, R₃ and R₄ are independently selected from hydrogen, hydroxy, and methoxy group;
R₅ and R₆ are methyl; and
R₇ is selected from hydrogen, methyl, amino, methylamino, dimethylamino, hydroxy and methoxy group.

61. The pesticide composition of claim 60,
wherein R₁ₐ, R_{1b}, R₂, R₃ and R₄ are independently selected from hydrogen, hydroxy, and methoxy;
R₅ and R₆ are methyl; and
R₇ is hydrogen.

62. The pesticide composition of claim 61, wherein said compound of formula (I) is selected from:
5-(3,4-dihydroxyphenyl)-6,7-dimethyl-5,6,7,8-tetrahydronaphthalene-2,3-diol;
(5R,6R,7R)-5-(3,4-dihydroxyphenyl)-6,7-dimethyl-5,6,7,8-tetrahydronaphthalene-2,3-diol;
4-(7-hydroxy-6-methoxy-2,3-dimethyl-1,2,3,4-tetrahydronaphthalen-1-yl)benzene-1,2-diol; and
4-((1R,2R,3R)-7-hydroxy-6-methoxy-2,3-dimethyl-1,2,3,4-tetrahydronaphthalen-1-yl)benzene-1,2-diol,
or combinations thereof.

63. The pesticide composition of claim 62, wherein said compound of formula (I) is (5R,6R,7R)-5-(3,4-dihydroxyphenyl)-6,7-dimethyl-5,6,7,8-tetrahydronaphthalene-2,3-diol.

64. The pesticide composition of claim 62, wherein said compound of formula (I) is 4-((1R,2R,3R)-7-hydroxy-6-methoxy-2,3-dimethyl-1,2,3,4-tetrahydronaphthalen-1-yl)benzene-1,2-diol.
